(19) United States
(12) Patent Application Publication
(10) Pub. No.: US 2010/0190199 A1
(43) Pub. Date: Jul. 29, 2010

(54) GPR30 ESTROGEN RECEPTOR IN BREAST AND OVARIAN CANCERS

(75) Inventors: Edward J. Filardo, East Grenwich, RI (US); Edmond Sabo, Providence, RI (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Rhode Island Hospital, Providence, RI (US)

(21) Appl. No.: 12/568,581

(22) Filed: Sep. 28, 2009

Related U.S. Application Data
(60) Provisional application No. 61/194,301, filed on Sep. 26, 2008.

(51) Int. Cl. C12Q 1/02

(52) U.S. Cl. 435/29

(57) ABSTRACT

A method of prognosis for ovarian or breast cancer patients is carried out by detecting GPR30 in a sample of tissue. An elevation in the level of GPR30 compared to a normal control level indicates presentation of late stage disease and indicates responsiveness of the tumor to hormonal therapy.

### RELATED APPLICATIONS

This application claims priority to U.S. provisional patent application 61/194,301, filed Sep. 26, 2008, the entire contents of which are hereby incorporated by reference.

### FIELD OF THE INVENTION

The present invention relates to the field of cancer diagnostics.

### BACKGROUND

The prognosis of ovarian carcinoma, the most lethal of all gynecological cancers, is grim, often with less than a two year life expectancy following surgical reduction of the tumor mass. Adjuvant therapy consists of platinum based drugs and taxanes as ovarian carcinoma is often metastatic at first detection and hormonal therapies targeted against the estrogen receptor, ER, yield favorable responses only in a small subset of patients.

### SUMMARY OF THE INVENTION

The invention provides new insight into the biological role of a novel estrogen receptor in ovarian cancer or breast cancer and refines the ability of medical professionals to identify subjects who will respond to hormonal therapy with aromatase inhibitors.

Specifically, the invention provides a method of prognosis for ovarian adrenocarcinoma patients, including detecting GPR30 in a sample of ovarian tissue following excision of a primary tumor, wherein an elevation in the level of GPR30 compared to a normal control level or over time indicates presentation of late stage disease.

The invention provides a method of prognosis for ovarian adrenocarcinoma patients, including detecting GPR30 and ER in a sample of ovarian tissue following excision of a primary tumor, wherein an elevation in the level of GPR30 and ER compared to a normal control level or over time indicates presentation of late stage disease. In one aspect of the above methods, late stage disease is stage II or III.

The invention provides a method for predicting survival time of an ovarian adrenocarcinoma patient, including detecting GPR30 in a tissue biopsy, wherein an increase in GPR30 level is correlated with a decrease in survival time. In one aspect of the above methods, the primary tumor or tissue biopsy includes serous, clear cell, endometrioid, or mucinous carcinoma cells.

The invention provides a method for predicting the presence of distant metastatic neoplastic disease in a subject diagnosed as including a primary tumor, the method comprising detecting an increase in a GPR30 level in a tissue sample obtained from the primary tumor, wherein the increase indicates that the subject is suffering from or at risk of developing a malignant tumor at an anatomical site distant from said primary tumor. In one aspect, this method further includes detecting an increase in the integrin α5β1 level in a tissue sample obtained from the primary tumor, wherein the increase indicates that the subject is suffering from or at risk of developing a malignant tumor at an anatomical site distant from the primary tumor. In another aspect of this method, the method includes detecting an increase in the matrix adhesion molecule SNAKA51 level in a tissue sample obtained from the primary tumor, wherein the increase indicates that the subject is suffering from or at risk of developing a malignant tumor at an anatomical site distant from the primary tumor.

The invention also provides a method of prognosis for the presence of distant metastatic neoplastic disease in patients, including detecting GPR30 in a sample of ovarian tissue following excision of a primary tumor, wherein an elevation in the level of GPR30 compared to a normal control level or over time indicates enhanced fibrillogenesis.

The invention provides a method of prognosis for the presence of distant metastatic neoplastic disease in patients, including detecting GPR30 in a sample of ovarian tissue following excision of a primary tumor, wherein an elevation in the level of GPR30 compared to a normal control level or over time indicates enhanced anchorage-independent growth.

The invention also provides a method of predicting the responsiveness of an ovarian tumor to hormonal therapy, including detecting GPR30 in a sample of ovarian tissue following excision of a primary tumor, wherein an elevation in the level of GPR30 compared to a normal control level or over time indicates an enhanced probability that hormonal therapy inhibits survival and/or spread of the tumor. In one aspect of this method, the hormonal therapy includes an aromatase inhibitor. For example, the ovarian tumor comprises serous, clear cell, endometrioid, or mucinous carcinoma cells. In another example, the tumor, e.g., breast tumor, is ER negative and GPR30-positive. Thus, the methods described herein are useful in delineating ER-negative breast cancer subtypes to inform a physician to choose the most appropriate therapy, e.g., chemotherapy or hormonal therapy.

The invention also provides a computer-readable medium having computer-executable instructions for performing a method. The process of transforming data pertaining to GPR30 expression/level in a tissue sample to a prognosis and/or choice of therapy is carried out as follows. First, at least one first variable associated with the level of GPR30 in sample of a patient is stored. Optionally, the levels are detected via binding to an antibody or another GPR30-specific ligand, and emitting a dectable, e.g., fluorescent, signal. At least one second variable associated with at least one reference level is stored. The patient's risk factor for the relevant clinical question, e.g., risk of metastasis or choice of therapeutic intervention (chemotherapy versus hormone therapy) is calculated as a function of at least the first and second variables. Finally, the risk factor is outputted.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

### BRIEF DESCRIPTION OF THE DRAWINGS

FIG. 1 is a schematic representation of GPR30 mediated signal transduction at the plasma membrane.

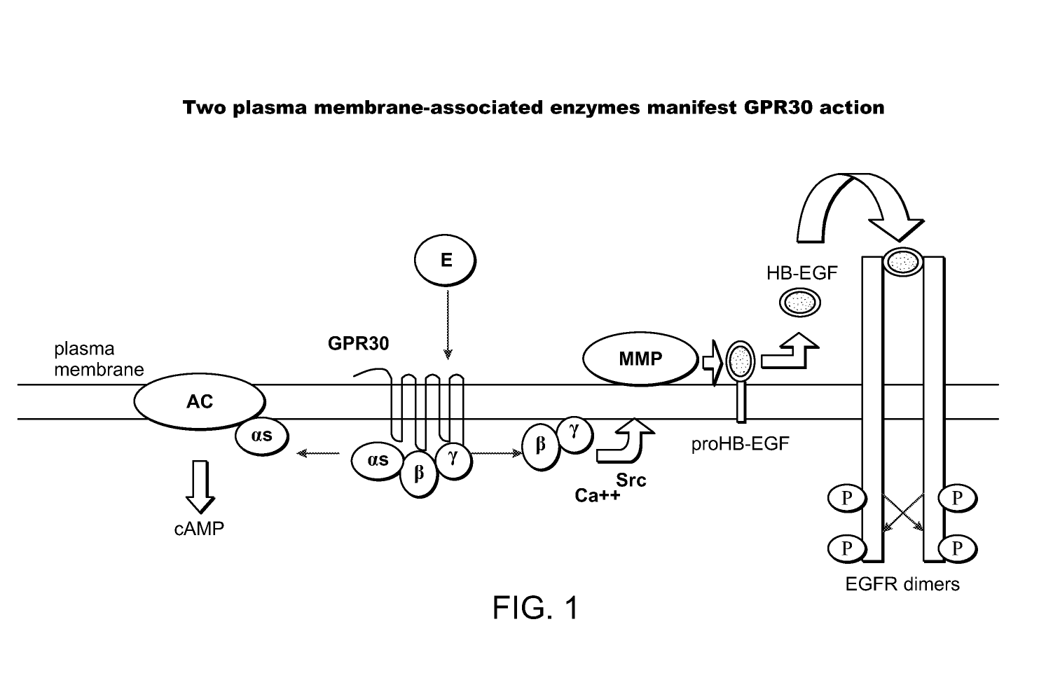

FIG. 2 is a schematic representation of seven transmembrane receptor (7TMR) structure and therapeutic targets.

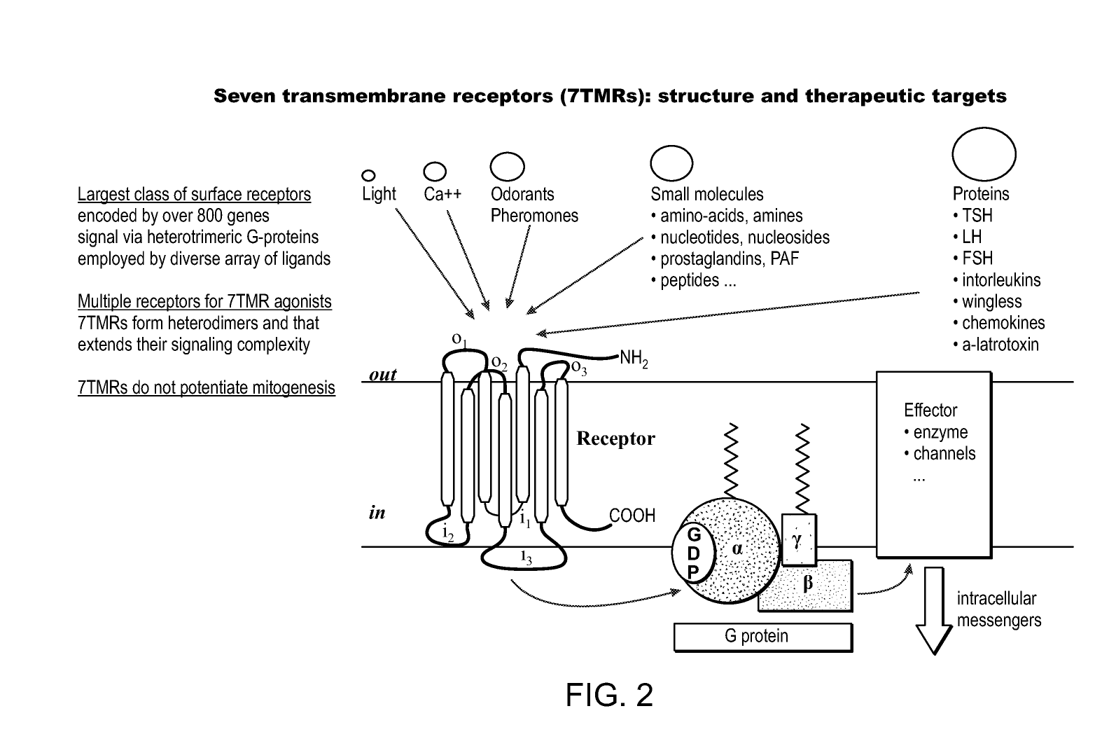

FIG. 3 is a schematic representation of the distribution of GPR30 in human breast carcinoma.

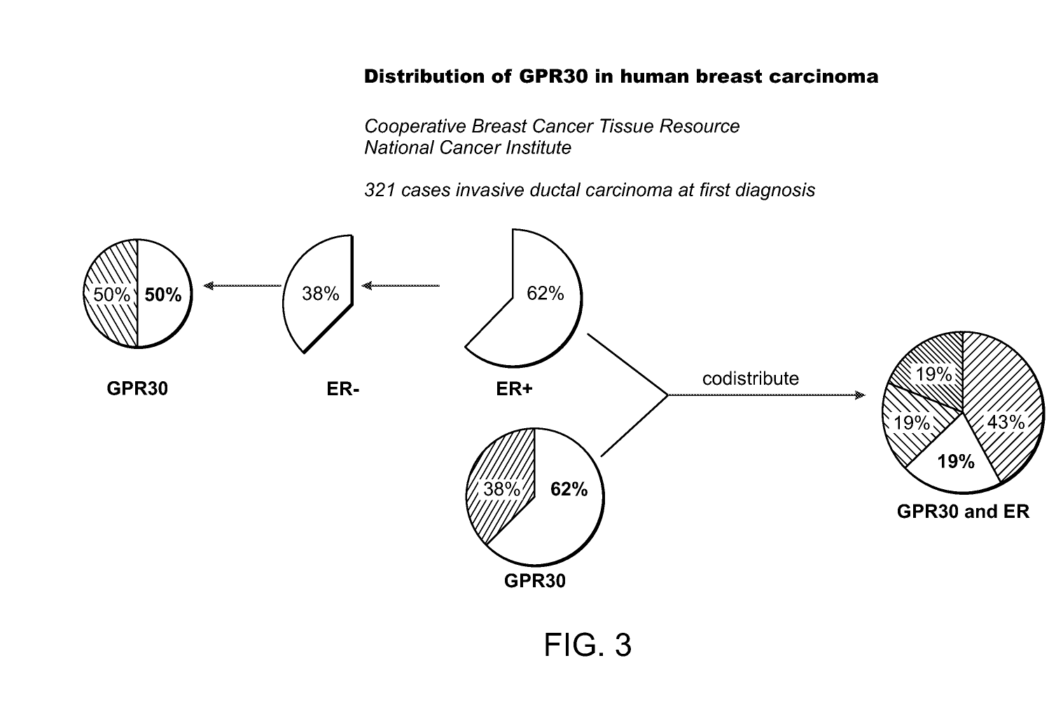

FIG. 4 is a schematic representation of GPR30 and ER expression in human ovarian adenocarcinoma. GPR30 was measured using peptide antibodies in tumor biopsy specimens of 57 human ovarian adenocarcinomas. GPR30 staining was scored in semiquantitative manner using a combined scoring system accounting for staining intensity and extent. The piechart on the left indicates number of specimens that were positive or negative for GPR30. Codistribution of GPR30 and ER was determined in 37 of these tumors that were selected for further analysis in a random manner, and the results are shown in the piechart on the right.

FIG. 5 is a series of photographs (magnified 200×) of archival, paraffin-embedded human ovarian tumor tissue immunostained with GPR30/GPER-1 or ER antibodies (top, ER+GPR30+ in extreme left column, ER+GPR− in left middle column, ER−GPR+ in right middle column, and ER−GPR− in extreme right column) with corresponding pie charts below quantifying the number of cells in each condition that are GPR30+ (light gray) or GPR30− (dark gray).

FIG. 6 is a graph of the relationship between GPR30 expression and cumulative survival in patients with serous ovarian adenocarcinoma. Representative examples of 37 ovarian tumor biopsy specimens that were immunostained with GPR30 and ER antibodies. The piecharts below contains codistribution data for GPR30 positive (green) or GPR30-negative (red) tumors in the ER-positive (n=13) or ER-negative (n=24) subgroups.

FIG. 7 is a series of graphs comparing expression of GPR30 and ER versus cumulative survival in patients with serous ovarian adenocarcinoma. Cumulative survival rates were plotted for biopsy specimens from 49 patients with serous ovarian tumors. Groups were divided into high and low expressors of either ER or GPR30 in their primary tumors. Bottom panel, cumulative survival rates are plotted for patients that are GPR30 positive and late stage. Biopsy specimens were immunostained with GPR30 peptide antibodies and scored based upon intensity and extent on a combined scale from 0-9. Cumulative survival data over 60 months of follow-up after tumor resection are plotted for patients that scored less than 4 or greater than or equal to 4.

FIG. 8 is a schematic representation of survival in serous ovarian adenocarcinoma as a function of cancer stage and GPR30.

FIG. 9 is a schematic representation of survival in serous ovarian adenocarcinoma as a function of cancer stage (left, early stage I, and right, late stage II+III) and GPR30.

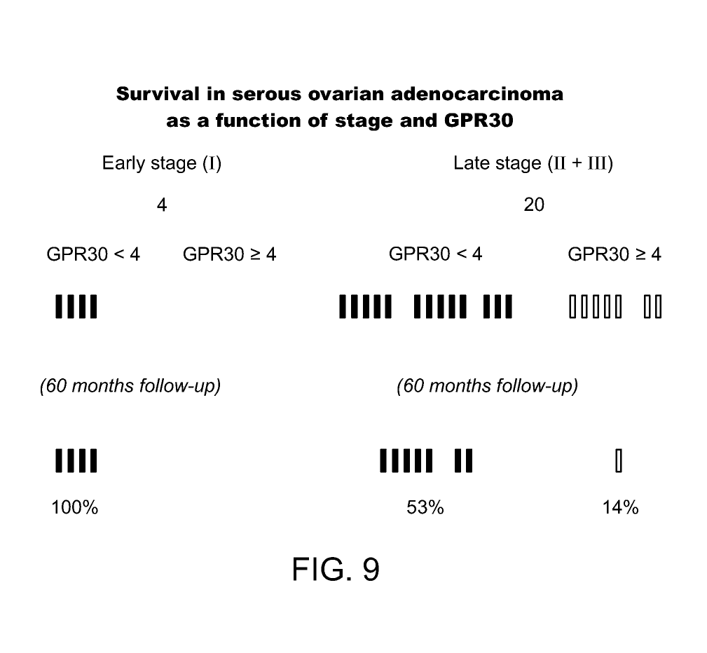

FIG. 10 is a graph of human SKBR3 breast cancer cell density versus time in which human SKBR3 breast cancer cells (Erα⁻, ERβ⁻, GPR30⁺) were treated with two different concentrations of EGF, E2, or negative control (untreated).

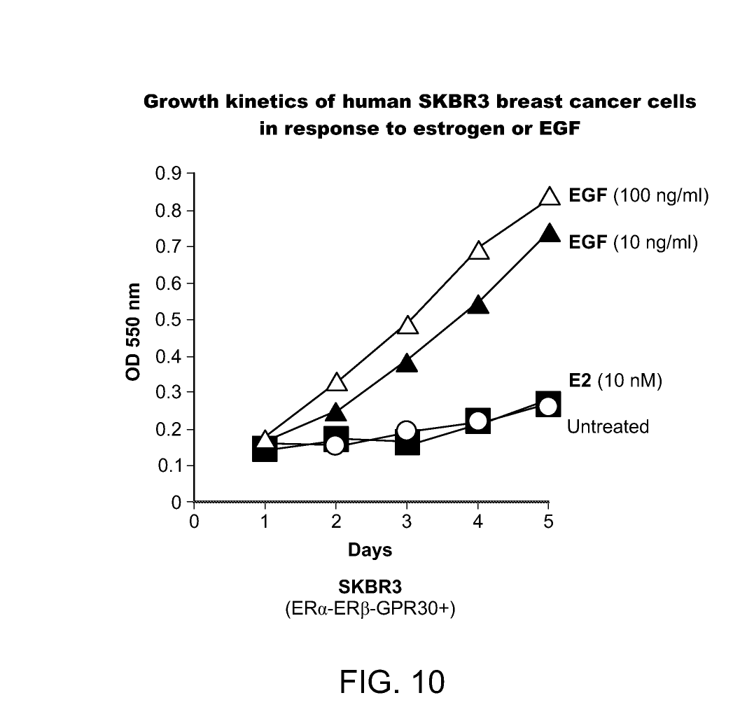

FIG. 11 is a schematic model of integrin activation.

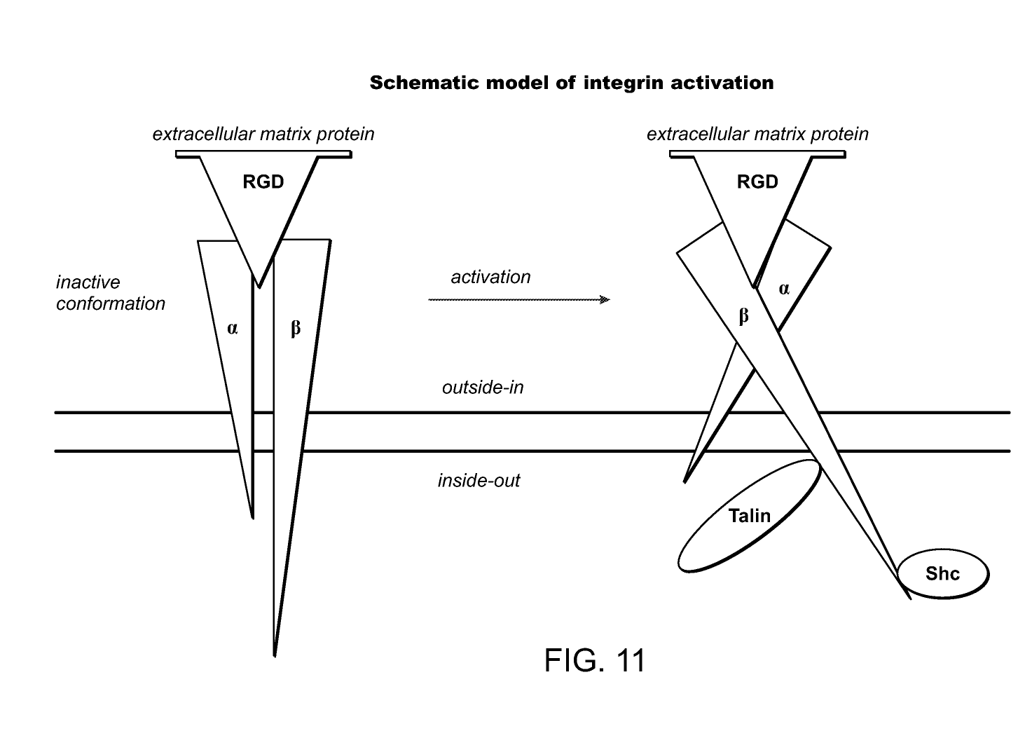

FIG. 12 is a series of photographs depicting 17β-E2-mediated SKBR3 breast cancer cell morphology changes induced by exogenous fibronectin application.

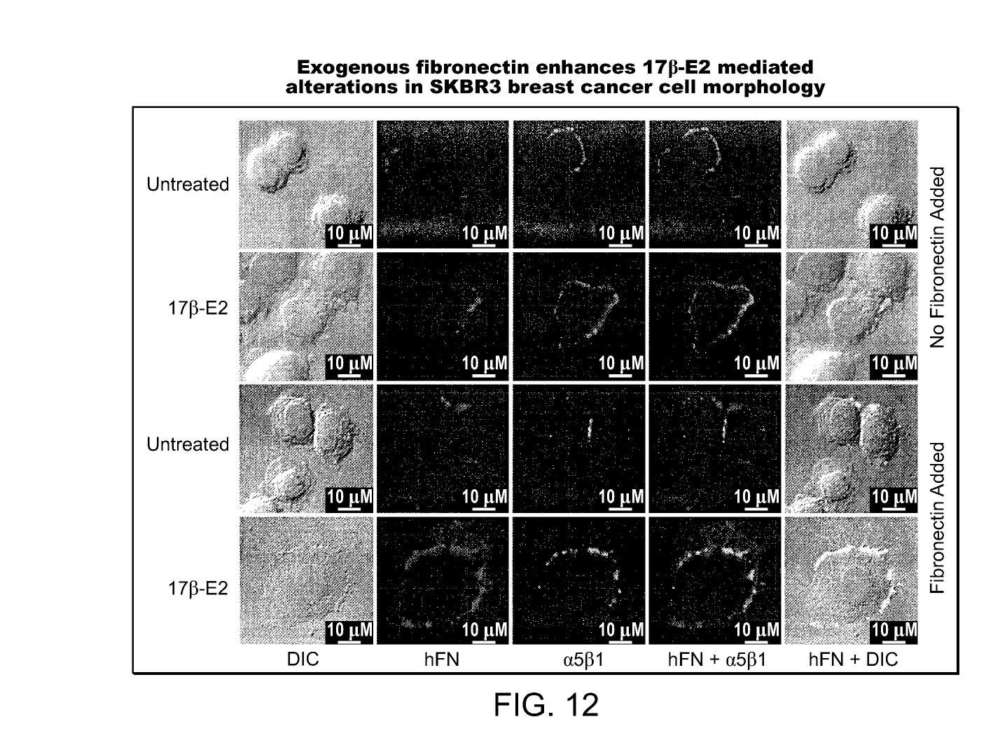

FIG. 13 is a series of photographs depicting the recruitment of fibronectin-occupied integrin α5β1 conformers to matrix adhesions following GPR30 stimulation.

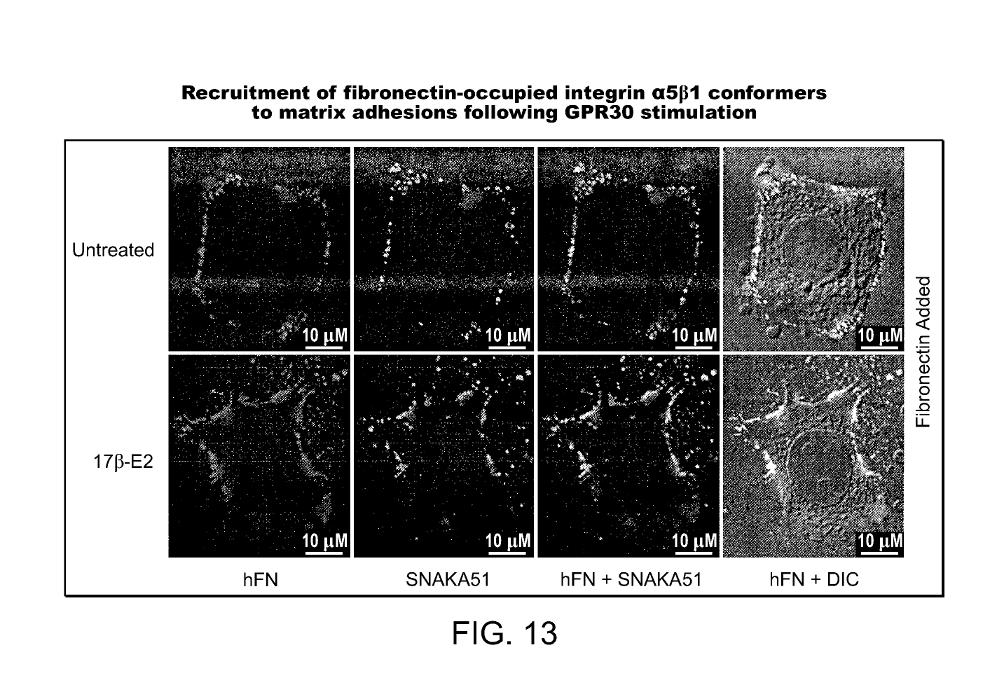

FIG. 14 is a series of photographs depicting gel electrophoresis of soluble and insoluble fibronectin protein isolated from cells transfected with either a vector control (left) or a GPR30 truncation mutant (right), which blocks E2-induced fibronectin assembly. SKBR3 (GPR30Δ154) or (vector) cells were fed exogenous rFN in the absence of stimulus or in the presence of 17β-E2, 17α-E2 or angiotensin II (ATII) for the indicated periods of time (hrs). DOC-soluble or insoluble proteins were immunoblotted with the rat FN-specific mAB, IC3.

FIG. 15 is a series of photographs depicting cells transfected with either a vector control (left) or a GPR30 truncation mutant (right), in which GPR30-mediated fibrillogenesis is disrupted.

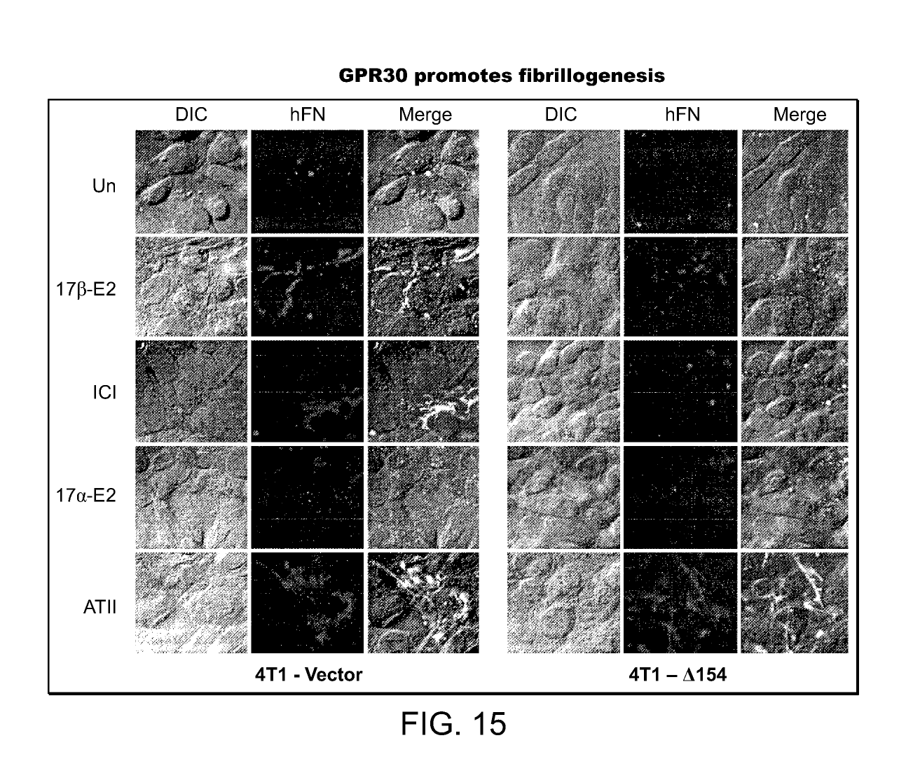

FIG. 16 is a series of photographs depicting cells transfected with either a vector control (left) or a GPR30 truncation mutant (right), which have been supplemented with fibronectin (bottom). Functional GPR30 enhances anchorage-independent growth.

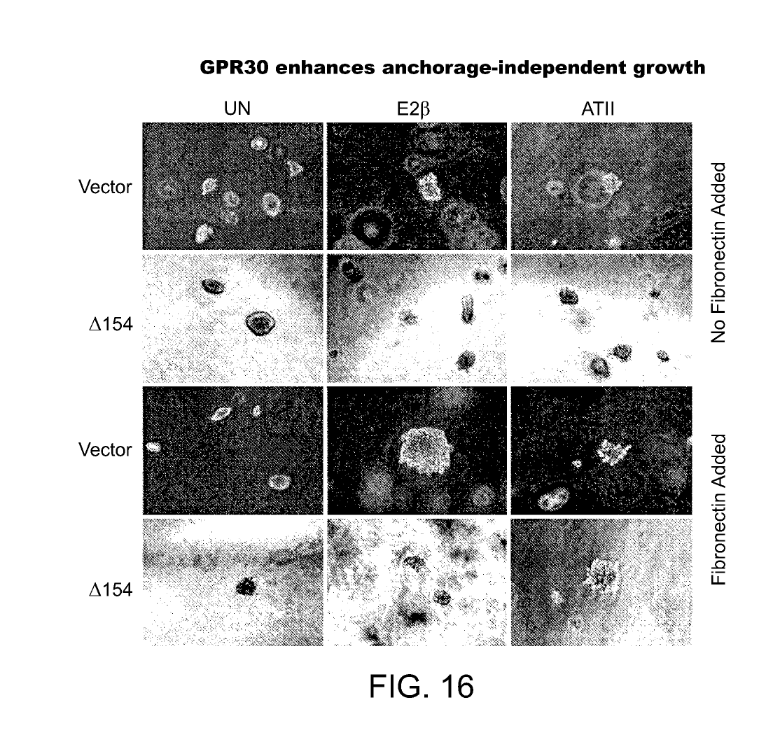

FIG. 17 is a series of photographs depicting cells transfected with vector control (left), the RDG integrin attachment site (RDG stands for the amino acid sequence Arg-Gly-Asp), or GPR30 truncation mutant (right).

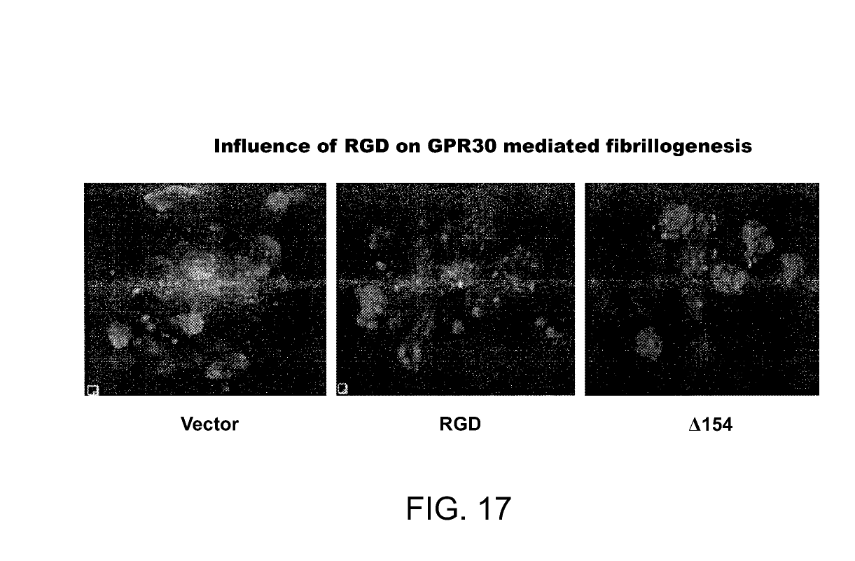

FIG. 18 is a schematic representation of the convergence of intracellular pathways linking the activities of estrogen, GPR30, non-receptor tyrosine kinases (SRC), adaptor proteins (SHC), RDG, integrins (α5β1), Fibril formation, Heparin-bound EGF (and the processing thereof, e.g. matrix metalloproteinase (MMP) cleavage of proHBEGF to release HBEGF), and the EGFR (and subsequent phosphorylation and dimerization thereof).

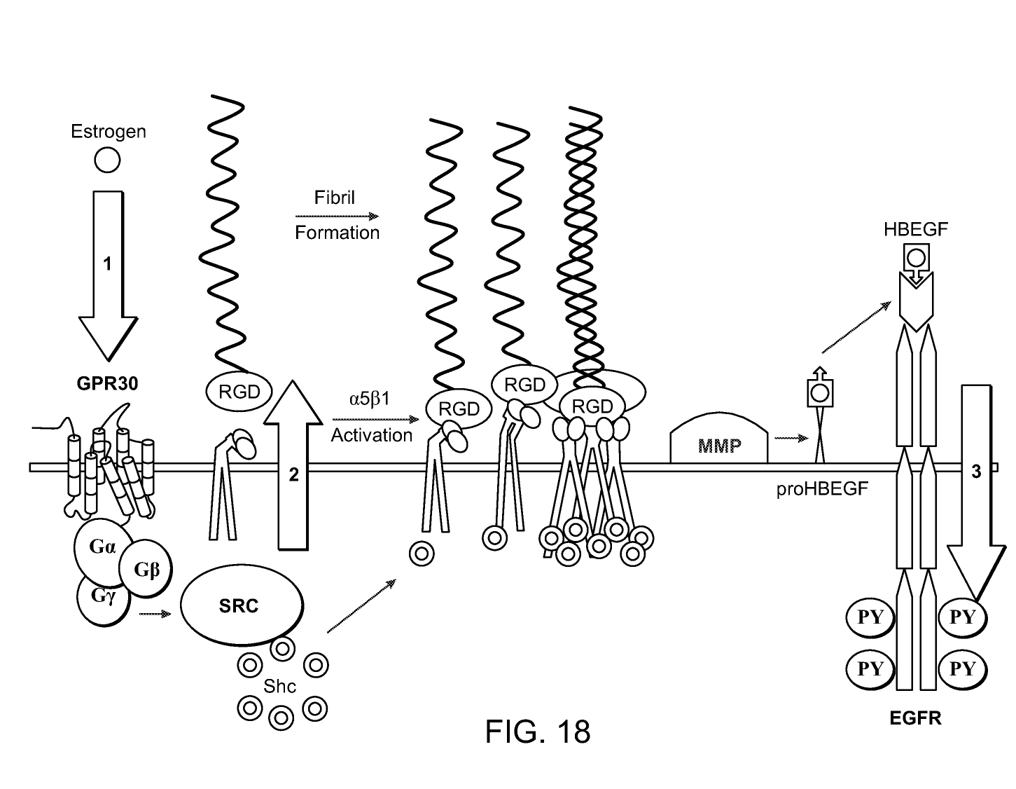

FIG. 19A-B is a series of photographs of Western Blot analysis of either EGFR or phosphorylated EGFR (pptyr) immunoisolated from SKBR3 cells (ERα−, ERβ−, GPR30+) which were transfected with HA-GPR30Δ154 or vector. (A) Equivalent amounts of total protein were blotted with HA antibodies. (B) Quiescent SKBR3 vector or HA-GPR30α154 transfectants were stimulated with E2 (1 nM), angiotensin II (1 nM) or EGF (10 ng/ml) for 5 min. Tyrosyl phosphorylation of erbB1 was measured in erbB1 immunoprecipitates by blotting with phosphotyrosine-specific mAB 4G10. erbB1 recovery was determined by reprobing with sheep erbB1 antibodies. The data demonstrate that GPR30 dominant interfering mutant selectively inhibits estrogen-mediated EGFR transactivation.

FIG. 20 is a line graph showing a Kaplan-Maier survival curve showing that GPR30 is linked to metastasis in a preclinical model. Murine 4T1 breast cancer cells (ER-negative, GPR30-positive) were injected intravenously. 4T1D cells express a dominant negative version of GPR30 that incapacitates the endogenous receptor. There is a statistically significant difference in the overall survival of mice receiving control 4T1 cells versus 4T1D cells (log rank, p=0.05). The mean overall survival time for 4T1 mice was 18 days. In contrast, the mean overall survival time for 4T1D mice was greater than 43 days. Extensive pulmonary metastases were observed in either treatment group at death.

### DETAILED DESCRIPTION

Patients with serous ovarian carcinoma are often face with a prognosis of less than a two-year life expectancy. Even though these tumors often express estrogen receptors (ERs), ER antagonists generally do not block tumor growth and progression. Consequently, standard adjuvant therapy for ovarian cancer patients consists of platinum-based drugs. However, recent studies have reported favorable responses with aromatase inhibitors that block estrogen biosynthesis. This observation implies that alternative estrogen receptors may facilitate estrogen-dependent growth and progression of ovarian carcinomas, findings that are encouraging since hormonal therapy is less toxic than chemotherapy. The transmembrane receptor, GPR30, is linked to specific estrogen binding, transactivation of the epidermal growth factor receptor, and the development of extra-mammary metastases. To determine the significance of GPR30 in ovarian cancer, we have evaluated its expression and relationship to disease progression variables. Using a semi-quantitative scoring system based upon staining intensity and extent, GPR30 expression was measured in normal ovarian epithelia and in adenocarcinoma. Preliminary analysis of 57 carcinomas indicated that GPR30 was expressed in two-thirds of cases and in 80% of serous tumors that lack ER (19 of 24). Of potential significance, a strong association was measured between tumors that express elevated GPR30 and overall patient survival at 5 years of follow-up (log rank=0.01). While GPR30 was uniformly positive in normal epithelia, its expression varies in carcinoma, in which 67% of the tumors evaluated (38 of 57) were positive. Among 24 ER⁻ tumors analyzed, 79% expressed GPR30. Unlike ER, which exhibited no significant association with tumor size, GPR30 positively associated with this progression parameter. In addition, a significant correlation between GPR30 and overall survival was observed in patients with serous adenocarcinoma (p=0.01, log rank). Over a five-year follow-up, 64% (11 of 17) of patients with a GPR30 score <4 were alive relative to 14% (1 of 7) of patients with a GPR30 score ≧4. Our results indicate that GPR30 independently assorts from ER and has a positive influence on ovarian tumor growth. Furthermore, our data implies that serous ovarian adenocarcinomas that express GPR30 represent more aggressive tumor variants that result in lower overall patient survival.

Estrogens are potent mitogens for tumors that arise from breast and ovarian epithelial tissue. While therapeutic decisions for the management of breast cancer are largely determined by measurement of estrogen receptors (ERs) in tumor specimens, the role of ERs in ovarian cancer is not clear. This is largely because few ovarian carcinomas express ER and those that do retain ERs, do not respond favorably to ER antagonism. As a result, ovarian cancer patients receive combination chemotherapy following surgery. However, this modality of treatment while effective, does not result in long term overall survival and ovarian cancer patients suffer extensive side effects from the commonly employed regimen of platinum-based drugs and radiation. The fact that endocrine therapy has measured some success in palliative settings, has minimal side effects, and has had good success in the treatment of breast cancer continues to provide impetus for studies aimed at identifying ovarian cancer patients that may respond to hormonal therapy.

Recent success using aromatase inhibitors to treat ER-positive breast cancer has renewed interest in testing the efficacy of these agents in patients with ovarian cancer. Phase II studies treating ovarian cancer patients with Letrozole, as second-line therapy for ER+ ovarian cancer, has resulted in stable disease as measured by the ovarian cancer serum marker CA-125 (Smyth et al. 2007. Clinical Cancer Research 13, 3617-3622; Raj Pandey, K. 2007. Lacet Oncol 8:678; Ramirez, P. T. et al. 2008. Gynecol Oncol. 110:56-59). These data indicate that the approach of inhibiting estrogen action may have a broader advantage over ER-targeted therapies, and raises the possibility that alternative estrogen receptors may promote estrogen action in ovarian cancer.

The Gs-coupled transmembrane receptor, GPR30 has been linked to specific estrogen binding, stimulation of adenylyl cyclase, and Gβγ-dependent release of heparan bound epidermal growth factor (HB-EGF). Moreover, GPR30 promotes gene transcription in ovarian cancer cells and proliferative effects in endometrial cancer cells. While the biological role of GPR30 has yet to be established, its expression has been linked to the development of advanced breast cancer and high-grade endometrial tumors. Recent studies into the mechanism by which GPR30 promotes EGFR transactivation indicate that integrin α5β1 is a necessary signaling intermediary in this pathway and thus, GPR30 provides a convergent signaling scheme that allows for coordinate release of growth factor and assembly of fibronectin matrices, critical events for tumor cell survival. These findings may, in part, explain the observation that 10% of patients with ER-negative breast tumors exhibit objective tumor regression and been cause for speculation that other mechanisms of tamoxifen action may contribute to its antitumor effects (Gradishar, W. I. and Jordan V. C. 1998. Endocrine Therapy of Breast Cancer. In Bland K. I. and Copeland, E. M. Eds. The Breast, 2^{nd} ed. Philadelphia, Pa.: W.B. Saunders and Co. pp. 1350-1372). The fact that ER antagonists promote GPR30 action in breast cancer cells, in part, explains why ovarian tumors are refractory to antiestrogen therapy.

Ovarian cancer is typically diagnosed in advanced stage and few tumors express ER (less than 30%). Consequently, ER antagonists are not suitable for treating ovarian cancer patients following tumor resection. However, recent studies have shown favorable responses in patients treated with aromatase inhibitors that have previously failed chemotherapy, indicating that some ovarian cancers may require estrogen for their growth and progression. Methods of the invention have determined that the seven transmembrane receptor, GPR30 specifically binds estrogen (Thomas P. et al. 2005. Endocrinol 146: 624-632), promotes rapidactivation of EGFRs (Filardo, E. J. and Thomas, P. 2005. TRENDS in Endocrin & Metab, 16:362-367) and is associated with advanced cancer (Filardo, E. I. et al. 2006. Clin Canc Res 12:6359-6366).

Estrogen promotes the development and homeostasis of the mammary gland, and the growth of tumors that arise from this tissue. It is widely accepted that estrogen manifests its physiological and pathophysiological actions through its interaction with specific receptors. Estrogen receptor (ER) α, and its structural homologue ERβ, belong to the nuclear steroid hormone family and function indisputably as hormone-dependent transcription factors. Blockade of estrogen binding sites on the ER, has proven to be an effective means to inhibit the growth of breast tumors expressing ER, and this modality of treatment remains the standard endocrine therapy for ER-positive tumors. While there is general concordance between ER expression and responsiveness to ER antagonism, as indicated in greater disease-free survival at 5 year follow-up for postmenopausal patients with ER-positive tumors receiving tamoxifen, roughly one-in-four patients do not respond to tamoxifen therapy. A variety of explanations have been offered to account for nonresponsiveness to ER antagonism, including: i) intratumoral heterogeneity in ER expression, ii) evolution of mutant ERs with reduced affinity for ER antagonists, iii) drug resistance, iv) partial receptor antagonism, and v) the presence or absence of trans-acting factors that influence ER functionality. These interpretations have prompted strategies better designed to assess ER activity and have served as rationale for the discovery and use of new endocrine agents with more complete ER antagonist activity. In addition, comarkers that better predict ER functionality have been identified for the purpose of selecting patients that will respond favorably to ER antagonists. For example, coexpression of the progesterone receptor, PR, the gene transcription of which is directly regulated by ER-dependent gene transactivation has prognostic value for determining favorable responses to tamoxifen. In addition, more complete ER antagonists, such as fulvestrant, are being assessed in clinical trials for patients with primary and advanced breast cancer.

All the ERs are widely distributed. The ERα is found in endometrium, breast cancer cells, ovarian stroma cells and the hypothalamus, while ERβ is found in kidney, brain, bone, heart, lungs, intestinal mucosa, prostate, and endothelial cells. Different estrogenic compounds have different binding affinities for alpha and beta ERs. While 17-beta-estradiol binds equally well to both receptors, estrone and raloxifene bind preferentially to the alpha receptor, and estriol and genistein to the beta receptor. The concept of selective estrogen receptor modulators is based on the ability to selective activate (or block) one type of ER or to promote ER interactions with different proteins such as transcriptional co-activator or co-repressor proteins. Additionally, the different estrogen receptor combinations respond differently to various antagonists, and some compounds have partially agonistic and antagonistic effects, depending on the tissue. For example, Tamoxifen, is an ER agonist in bone and uterus, but antagonist in breast tissue, and is therefore used in breast cancer treatment.

The existence of alternative estrogen receptors, whose action is not blocked by ER antagonists, or possibly stimulated by ER antagonists, has also been offered as a possible explanation for tamoxifen nonresponsiveness. Studies in animal and cell models have long indicated that estrogen manifests physiological actions and biochemical effects inconsistent with its known genomic mechanism of action. For instance, estrogen induces EGF-like activity in female reproductive tissue and likewise activates biochemical signals typically associated with EGFRs. Estrogen also stimulates second messenger signaling characteristic of seven transmembrane-spanning receptors (7TMRs), including activation of calcium, cAMP and inositol triphosphate. The orphan 7TMR, GPR30, is linked to estrogen-mediated stimulation of adenylyl cyclase, release of heparan bound (HB)-EGF from the surface of breast cancer cells, and specific estrogen binding. GPR30 acts independently from ERα and ERβ, and triggers estrogen-dependent EGFR action. GPR30 plays an important role in breast cancer biology since it provides a mechanism by which estrogen promotes EGF-like effects. Breast tumors that lack ERs may remain estrogen responsive by employing GPR30. This concept is particularly intriguing for patients receiving endocrine therapy, since “partial” (tamoxifen) and “pure” (faslodex) ER antagonists behave similarly to estradiol, and are capable of triggering EGFR activation in breast cancer cells.

### Seven-Transmembrane Receptors (7TMRs) in Rapid Estrogen Signaling

The observation that estrogen promotes rapid biochemical actions predates the first report describing the existence of specific binding activity for estrogen in extracts from rat female reproductive tissue. Early studies demonstrated that intrauterine administration of estrogen in rats resulted in a rapid rise in intracellular cAMP. However, the mechanism by which estrogen produced cAMP was largely ignored after the isolation of an estrogen receptor (ER) with structural characteristics of a hormone-inducible transcription factor. Subsequent experiments in vitro confirmed that estrogen generates cAMP as a result of its ability to stimulate adenylyl cyclase. Observations that estrogen also stimulates Ca²⁺, inositol triphosphate, and heterotrimeric G proteins indicated that a seven-transmembrane-spanning receptor (7TMR) might be implicated in rapid estrogen signaling. Rapid estrogen signaling was linked to the orphan receptor, GPR30. The amino acid sequence of GPR30 reveals a serpentine, heptahelical structure characteristic of 7TMRs.

### Endocytic Fates of 7TMRs

TMRs promote intracellular signals and biological responses as a result of changes in receptor activation and inactivation states. Binding of cognate ligand to 7TMRs, induces allosteric changes in their structure causing the 7TMR to function as a GDP/GTP exchange factor promoting the loading of GTP into the active site of the Gα-GTPase subunit and its subsequent release from the Gβγ subunit components of the receptor-associated Gαβγ heterotrimer. Both Gα− and Gβγ− proteins promote intracellular signaling events that ultimately provide feedback that attenuates 7TMR signaling and protects from receptor overstimulation. This process is initiated by receptor phosphorylation, and is often but not exclusively mediated by β-arrestins that bind phosphorylated 7TMRs resulting in the physical uncoupling of heterotrimeric G proteins from 7TMRs and their recruitment to endocytic machinery often via clathrin-coated pits. Once internalized, 7TMRs are sorted into two separate trafficking patterns. Either they are dephosphorylated, resensitized and recycled back to the plasma membrane or they are marked for destruction in lysosomes, a process associated with ubiquitination of 7TMR, or associated proteins, such as β-arrestin. Based upon this interaction, receptors are divided into two classes. “Class A” receptors (β1-adrenergic receptors) form transient interactions with β-arrestin. These receptors do not require β-arrestin for internalization and recycle relatively rapidly. In contrast, “class B” receptors (angiotensin II and vasopressin receptors), bind β-arrestin more avidly and internalize together with it. These receptors recycle more slowly, which is reflected by their retention in endosomes or proteolysis in lysosomes.

Estrogen and EGF are important paracrine regulators of mammary gland development and homeostasis. Transactivation of EGFR by GPR30 has significance in breast cancer. The data described herein directly implicates GPR30 in breast tumor progression. These data positively associate GPR30 expression in primary tumors with increased tumor size and metastases.

### Antibody Specificity

The present invention features antibodies or fragments thereof that specifically bind to GPR30. Such antibodies include monoclonal and polyclonal antibodies, that specifically bind to antigenic sequences within the GPR30 polypeptide (Accession Numbers CAG46541, CAG46456, NP_{—}001026852, NP_{—}001496, NP_{—}084047, or XP_{—}355659), which is described, for example, in Filardo and Thomas, Trends Endocrinol. Metab. 16: 362-7, 2005), and Filardo, J. Steroid Biochem. Mol. Biol. 80:231-8, 2002, which are hereby incorporated by reference. Epitope binding specificity of GPR30-specific monoclonal antibodies are defined by a sequence of 8, 10, 12, 15, 18, or 20 consecutive amino acids of a GPR30 protein sequence. For example, these epitopes correspond to consecutive residues in the sequence of GENBANK accession number CAG46541 (with the exception of the first 24 residues, which represent a signal sequence that is cleaved during post-translational processing).

Human GPR30 is encoded by the following amino acid sequence (NCBI Accession No. CAG46541 and SEQ ID NO: 1, residues 1-24 correspond to the signal peptide):

| | |
|---|---|
| 1 | mdvtsqargv glemypgtaq paapnttspe lnlshpllgt alangtgels ehqqyviglf |
| 61 | lsclytiflf pigfvgnili lvvnisfrek mtipdlyfin lavadlilva dslievfnlh |
| 121 | eryydiavlc tfmslflqvn myssvffltw msfdryiala ramrcslfrt khharlscgl |
| 181 | iwmasvsatl vpftavhlqh tdeactcfad vrevqwlevt lgfivpfaii glcyslivrv |
| 241 | lvrahrhrgl rprrqkalrm ilavvlvffv cwlpenvfis vhllqrtqpg aapckqsfrh |
| 301 | ahpltghivn laafsnscln pliysflget frdklrlyie qktnlpalnr fchaalkavi |
| 361 | pdsteqsdvr fssav |

Human GPR30 protein contains 7 transmembrane domains which are encoded by the following amino acid sequence fragments:

| From | To | Length | Domain |
|---|---|---|---|
| 1 | 62 | 62 | Extracellular (Exo I) |
| 63 | 84 | 22 | TM1 |
| 85 | 96 | 12 | Cytoplasmic |
| 97 | 120 | 24 | TM2 |
| 121 | 132 | 12 | Extracellular (Exo II) |
| 133 | 153 | 21 | TM3 |
| 154 | 175 | 22 | Cytoplasmic |
| 176 | 194 | 19 | TM4 |
| 195 | 220 | 26 | Extracellular (Exo III) |
| 221 | 236 | 16 | TM5 |
| 237 | 259 | 23 | Cytoplasmic |
| 260 | 280 | 21 | TM6 |
| 281 | 306 | 26 | Extracellular (Exo IV) |
| 307 | 327 | 21 | TM7 |
| 328 | 375 | 48 | Cytoplasmic |

The antigenic sequences are located throughout the protein sequence, e.g., in the C-terminus, N-terminus, Exodomain II, or Exodomain IV of the GPR30 polypeptide. The antibody specifically binds an antigen in the following peptide sequences: HERYYDIAVLC (SEQ ID NO: 2; in Exodomain II), KQSFRHAHPLTGHIC (SEQ ID NO: 2; in Exodomain IV), CAVIPDSTEQSDVRFSSAV (SEQ ID NO: 4; in C-terminus), or MDVTSQARGVGLEMYPGTAQPAAC (SEQ ID NO: 5; in N-terminus). Preferred antibodies bind to domains that are extracellular or cytoplasmic, rather than embedded in the cell membrane (TM). For example, the antibodies are generated using peptides 15-25 residues in length (e.g., 18-mers, 19-mers, 20-mers, 21-mers) located in the Exo I, II, III, or IV domains (sequence coordinates described above) or in either of the cytoplasmic domains (sequence coordinates also described above).

The antibodies described herein are used, for example, to detect the presence of a tumor cell in a biological sample (e.g., primary tissue biopsy, archival tissue (frozen or formalin-fixed), cultured cells or cell lines, or biological fluid (e.g., plasma or blood)) using any method known in the art including immunohistochemical methods or ELISA assays. The identification of cells expressing GPR30 identifies the biological sample as containing a tumor cell, such as a tumor cell of the reproductive system (e.g., breast tumor cell, ovarian tumor cell, or uterus tumor cell). Moreover, GPR30 monospecific antibodies are useful for screening for novel estrogen-based therapies for breast or ovarian cancer. Furthermore, the expression of GPR30 in a primary breast cancer predicts the risk or presence of distant metastases. The data indicate that the presence of GPR30 positive primary tumors have a higher likelihood of presenting with distant metastases compared to individuals with GPR30 negative primary tumors.

Prior to the invention, estrogen receptors, ERα and ERβ, were the primary clinicopathological variable used for determining adjuvant therapy for patients with primary and advanced breast cancer. Antagonists to such receptors (e.g., tamoxifen) activate GPR30. Thus, GPR30 antibodies allow clinicians to refine the assignment of patients for appropriate adjuvant therapy since the identification of such tumors using the GPR30 antibodies described herein would identify patients in need of therapeutic strategies that differ from those based on ERα or β alone.

### GPR30 as a Predictor of Breast Tumor Metastasis

Expression of GPR30 in primary breast tumors is strongly correlated with the presence of distant metastasis (p-value=0.014). Measurement of GPR30 in human breast tumors using GPR30 peptide antibodies, polyclonal or monoclonal, by immunohistochemical or biochemical analysis, is used to determine whether a patient with primary breast cancer harbors metastatic seeds (visible or occult). The results of this measurement are used to determine decisions/options for the treatment of primary breast cancer. The data also has prognostive value in the detection of GPR30 levels, e.g., using GPR30 peptide-specific antibodies. Increased GPR30 expression in preneoplastic disease projects progression to frank neoplasia.

### Coordinate Regulation of Estrogen-Mediated Fibronectin Matrix Assembly and Epidermal Growth Factor Receptor Transactivation by the G Protein-Coupled Receptor, GPR30

The data described herein elucideates the mechanism by which GPR30 facilitates the implantation of metastatic breast tumor cells. Estrogen promotes changes in cytoskeletal architecture not easily attributed to the biological action of estrogen receptors, ERα and ERβ. The Gs-protein-coupled transmembrane receptor, GPR30, is linked to specific estrogen binding and rapid estrogen-mediated release of heparan bound epidermal growth factor (HB-EGF). Marker rescue and dominant interfering mutant strategies were used to show that estrogen action via GPR30 promotes fibronectin (FN) matrix assembly by human breast cancer cells. Stimulation with 17β-estradiol or the ER antagonist, ICI 182,780, resulted in the recruitment of FN-engaged integrin α5β1 conformers to fibrillar adhesions and the synthesis of FN fibrils. Concurrent with this cellular response, GPR30 promotes the formation of Src-dependent, Shc-integrin α5β1 complexes. Function-blocking antibodies directed against integrin α5β1 or soluble RGD (Arg-Gly-Asp) peptide fragments derived from FN specifically inhibited GPR30-mediated EGFR transactivation. Estrogen-mediated FN matrix assembly and EGFR transactivation were similarly disrupted in integrin β1-deficient GE11 cells, whereas reintroduction of integrin β1 into GE11 cells restored these responses. Mutant Shc (317Y/F) blocked GPR30-induced FN matrix assembly and tyrosyl phosphorylation of erbB1. Relative to recombinant wild-type Shc, 317Y/F Shc was more readily retained in GPR30-induced integrin α5β1 complexes, yet this mutant did not prevent endogenous Shc-integrin α5β1 complex formation. The results indicate that GPR30 coordinates estrogen-mediated FN matrix assembly and growth factor release in human breast cancer cells via a Shc-dependent signaling mechanism that activates integrin α5β1. The data support the mechanism that GPER-1 facilitates tumor metastasis by coordinating growth factor release and fibronectin matrix assembly, key events for successful tumor implantation at a distant site.

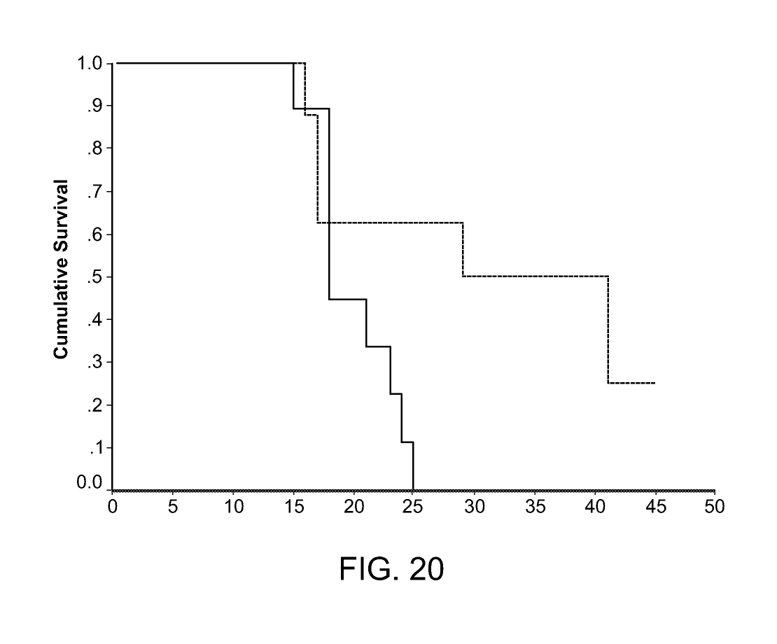

FIG. 20 shows a Kaplan-Maier survival curve. Murine 4T1 breast cancer cells (ER-negative, GPR30-positive) were injected intravenously and survival evaluated. 4T1D cells express a dominant negative version of GPR30 that incapacitates the endogenous receptor. In the 4T1D (GPR30 inactivated) group, 9 out of 9 mice have died by the end of experiment (Median Survival Time=18 days, Mean Survival Time±SEM=20±1.12). In the 4T1 (GPR30 intact) group, 6 out of 8 mice have died by the end of study (Survival rates at the end=25%, Median Survival Time=29 days, Mean Survival Time±SEM=31.38±4.35). Comparison between the survival rates: p=0.04 (Log Rank test).

GPR30 serves a dual purpose in that it regulate integrin α5β1-dependent FN matrix remodeling and also promotes release of EGF-like growth factors to ensure cellular survival during estrus cycle-dependent remodeling of the mammary gland. Breast tumors that maintain GPR30 employ this integrin-dependent autocrine loop to their advantage. The fact that ER antagonists also act through GPR30 further indicates a mechanism by which ER antagonists may actually facilitate tumor growth and invasion.

Thus, an important consideration in identifying a patient for adjuvant therapy is detecting a GPR30-positive tumor in the patient. Tamoxifen is a partial ER antagonist. The presence of a GPR30-positive tumor indicates that a treatment regimen comprising ER antagonists such as tamoxifen is not indicated or recommended. Testing for GPR30 is important in determining whether a patient receives chemotherapy in contract to hormonal therapy. Given the biology of estrogen receptor-negative breast cancer in various racial and ethnic groups, segregation of patients based on GPR30 expression becomes more important. For example, African-Americans have a greater chance of having ER-negative breast cancer compared to other groups.

### ER-Negative Breast Cancer in Various Racial and Ethnic Groups

Significant disparities exist with regard to breast cancer incidence and mortality among racial and ethnic groups for which clinical data are available. Specifically, while the incidence of breast cancer in African-American women (AA) is lower than that of non-Hispanic whites (NHW), the overall breast cancer mortality rate is higher among AA. Breast cancers exhibit heterogeneity at the clinical, microscopic, and molecular level. Breast tumors are staged clinically based on tumor size, presence of nodal metastasis and distant metastasis (TNM classification). An important classification of breast tumors is based on the presence or absence of the estrogen receptor. While the majority of breast cancers are ER-positive, approximately 30% are ER-negative. The presence of the estrogen receptor and the growth factor receptor Her 2 allows oncologists to choose targeted therapy. Patients with ER-positive breast cancer receive hormonal therapy using either tamoxifen or aromatase inhibitors and have a better prognosis. The presence of Her 2 allows the targeting of breast tumors with the HER 2 receptor-specific drugs, herceptin or lapatinib. However, the treatment of patients with tumors that are both ER-negative and Her 2-negative (HER 2−) is a challenge. Given the lack of targeted therapies, the main options for ER−/HER 2− breast cancer patients are limited to conventional cytotoxic chemotherapy

The methods are useful to segregate subtypes of ER-negative breast cancers, e.g., GPR30 expression, in order to determine the most appropriate treatment, particularly among racial and ethnic groups. For example, African-American women have a higher incidence of ER-negative tumors. The information will be crucial in developing early detection and intervention strategies. The identification of the subtypes or heterogeneity that exist within ER-negative breast cancers

### Therapeutic Target for Breast Cancer

The transmembrane receptor, GPR30 (a.k.a., GPER-1), is linked to specific estrogen binding, rapid estrogen signaling and breast cancer metastasis. Estrogen action through GPR30 allows for breast tumor cell survival, in contrast to breast tumor cell proliferation. Estrogen acts through GPR30 to promote the rapid release of preformed growth factors that are tethered to the surface of breast cancer cells. The data described herein provide a mechanism by which GPR30 triggered the release of epidermal growth factor (EGF) polypeptides from the surface of breast cancer cells. The results indicated that the growth factors did not promote cellular growth. EGF-related factors are also important in other cellular activities such as cellular survival, and the data revealed that estrogen action through GPR30 had a more profound effect on tumor cell survival. GPR30 was found to promote the assembly of a provisional extracellular matrix—a crucial event in cellular survival. More specifically, release of growth factor by GPR30 required the activation of a latent adhesion receptor (integrin α5β1).

Activation of integrin α5β1 by GPR30 is a significant event, because it provides a way for invading cells to gain hold once they metastasize to tissues distant to the primary breast cancer. This event happens, because activated integrin α5β1 converts soluble plasma protein fibronectin into an insoluble cage. The breast cancer cells use this to adapt to a new environment.

In general, about two-thirds of all breast cancer cases involve tumors that retain expression of estrogen receptors (ER). They are presumed to proliferate in response to estrogen produced by the patient. Consequently, patients with ER-positive tumors receive hormonal agents (known as ER antagonists) that act by blocking the proliferative effects of estrogen promoted by the ER. As a result, the capacity of breast cancers to grow is reduced. The development of drugs targeting GPR30 is an important step in controlling breast cancer, because this estrogen receptor is not promoting estrogen-dependent growth but is critical in promoting breast tumor cell survival.

ER-positive breast tumor patients are often treated with aromatase inhibitors such as tamoxifen that block estrogen biosynthesis. The discovery that GPR30 represents yet another estrogen receptor with biological significance for breast cancer furthers the argument that aromatase inhibitors would effectively block estrogen action at both types of estrogen receptors.

### Identification of Patients for Adjuvant Therapy

GPR30, a 7 transmembrane (TM) G-protein coupled receptor (GPCR) is involved in estrogen-mediated cell signal transduction and estrogen binding. The GRP30 receptor acts independently from estrogen receptors, ERα and ERβ. About two-thirds of all breast cancers contain elevated levels of estrogen receptors compared to nontumor cells. These tumors are characterized as estrogen receptor positive (ER+). Patients with ER+ tumors are candidates for adjuvant hormone therapy. Antibodies and other ligands specific for GPR30 formulated as detectable probes are used to identify cancer patients for hormone adjuvant therapy.

Adjuvant hormone therapy deprives cancer cells of the female hormone estrogen, which some endocrine-responsive cancer cells need to proliferate. In addition to surgery or radiation as a primary therapy, adjuvant hormone therapy with, e.g., an anti-hormone drug such as tamoxifen, inhibits proliferation of residual tumor cells, prevents the original cancer from returning, and/or prevents the development of new cancers in the other locations or tissues. Identification of this subset of patients is critical to formulating an effective treatment regimen.

The GPR30-specific ligands described herein are also useful as tools for prognosis. ER+ tumors tend to grow less aggressively than ER− tumors. Therefore, patients with ER+ tumors (increased levels of GPR30 as determined using GPR-specific monoclonal antibodies described herein) have a better prognosis than those with lower levels of GPR30.

A tissue sample, e.g., a resected tumor or biopsy sample, is contacted with a GPR30-specific antibody and the level of GPR30 is determined and compared to a control value. The control value is a level of GPR30 that is associated with tumors of the reproductive system (e.g., breast cancer, ovarian cancer) that are not hormone receptor positive. An increase in the level of GPR30 compared to the control indicates that the patient from which the sample is obtained is a good candidate for hormone adjuvant therapy.

### Diagnostic Reagents

The invention also provides a diagnostic reagent pack or kit containing one or more containers filled with one or more of the agents of the invention. Reagents, e.g., antibodies that specifically bind peptides containing an antigenic sequence of the Exodomain II of GPR30 (e.g., HERYYDIAVLC; SEQ ID NO: 6) for carrying out the diagnostic or prognostic assay may be packaged together as a kit. For example, the antibody is immobilized on a solid phase and packaged together with other reagents suitable for detecting the peptide-antibody complexes. For example, enzyme-conjugated reagents may be included. Antigenic peptides that bind specifically to the antibody may also be included as a standard or control reagent. For example, the solid phase component of the kit onto which an antibody is immobilized is an assay plate, an assay well, a nitrocellulose membrane, a bead, a dipstick, or a component of an elution column. The kit may also contain a second antibody or other detectable marker. The second antibody or marker is labeled, e.g., using a radioisotope, fluorochrome, or other means of detection. The pack or kit can be labeled with information regarding the sequence of execution (e.g., obtaining a biological sample, contacting with a peptide containing an antigenic sequence, and detecting the presence or absence of antibodies specific to the peptide in the biological sample), or the like. The pack or kit can be a single unit assay or it can be a plurality of unit assays. For the purpose of this invention, unit assays is intended to mean materials sufficient to perform only a single assay.

### Identification of GPR30 Inhibitors

Therapeutic intervention for GPR30-overexpressing tumors includes administration of a GPR30 inhibitor to reduce or prevent metastasis.

Expression of GPR30 is required for rapid biochemical signaling events and subsequent cell biological effects measured in breast cancer cells. The monoclonal GPR30 peptide-specific antibody described herein is also useful to purify GPR30 protein for the purpose of determining the minimal binding sites necessary to support estrogen action as well as to identify inhibitors of estrogen/GPR30 binding. These mABs are useful in solid state assays for the purpose of measuring the activity of GPR30 for estrogen analogues, paralogues, homologues, other estrogen mimetics, xenoestrogens, anti estrogens, estrogen receptor antagonists, and selective estrogen response modifiers (SEMS). These assays employ traditional radioreceptor binding assays as well as binding assays for fluorescently labeled estrogen-like molecules. Furthermore, these antibodies are used for the measurement of receptor activity as measured by allosteric changes in receptor structure.

To screen for a GPR30 inhibitor, a cell expressing GPR30, e.g., COS cells that express recombinant GPR30-GFP, are contacted with one or more candidate compounds in the presence and absence of estrogen. A reduction in the level of estrogen binding in the presence of the candidate compound compared to the level in the absence of the candidate compound indicates that the candidate compound inhibits estrogen binding to GPR30. Similar binding studies are carried out using ERα and ERβ (e.g., using COS cells transfected with ERα-GFP or ERβ-GFP). Inhibition of binding in the GPR30 system but not the ERα/ERβ system indicates that the candidate compound specifically inhibits binding of estrogen to GPR30 (as compared to other estrogen receptors). 7TMRs undergo allosteric changes in receptor structure in response to binding agonist, antagonists and inverse agonists. Altered antibody binding therefore predicts a change in receptor structure and function. Similarly, candidate compounds are tested for their ability to inhibit or influence/alter the binding profile of a GPR30-specific antibody, e.g., an antibody with a binding specificity for a particular epitope of GPR30 (SEQ ID NO: 2, 3, 4 or 5). Those candidate compounds that inhibit estrogen binding and/or binding of a GPR30-specific antibody are further tested for their effect on intracellular calcium mobilization and PI-3 kinase (PI3K) activation. Binding assays and signal transductions assays such as calcium mobilization and PI3K measurements are well known in the art, e.g., Bologa et al., 2006, Nat. Chem. Biol 2:207-212. A reduction in the level of calcium mobilization and/or PI3K activation in the cell compared to the level of mobilization observed with estrogen indicates that the candidate compound inhibits GPR30 activity in a cell and is useful to inhibit GPR30-mediated tumor growth and progression and/or GPR30-mediated metastasis.

Another strategy involves using two GPR30 antibodies to measure GPR30 activity by resonance energy transfer such as Fluorescence Resonance Energy Transfer (FRET) or Bioluminescence Resonance Energy Transfer (BRET). For example, a FRET assay is carried out as follows. Each antibody is coupled to a different fluorochrome. One fluorochrome emits at a higher energy than the excitation energy of the second fluorochrome. These antibody-fluorochrome conjugates are then applied to freshly isolated tumor specimens (not fixed) and exposed to a light source that activates fluorochrome 1 but not 2. Fluorochrome 2 absorbs light reemitted from fluorochrome 1. The amount of energy transferred is a function of distance. This assay measures a change in the distance between the site occupied by the first antibody and the second antibody, and thus indicates that GPR30 has changed its shape and thus its activity. Evidence of an allosteric change is useful for assessing how active GPR30 is in a tumor specimen, and thereby influence decisions regarding therapy.

For example, mABs are used to tag two different structural regions of the extracellular face of the receptor, e.g., exo II and exo IV. mAB to exo II is tagged with a fluor that absorbs in the blue range and reemits in the green range. Exo IV mAB is tagged with a fluor that absorbs light in the green range and reemits in the red range. In a static state, blue light absorbed by exo II mAB is reabsorbed as green light by the exo IV mAB and reemited as red light. The efficiency by which the energy is transferred (Fluorescence Resonance Energy Transfer) is a function of the distance between the two fluors. If the receptor binds its ligand (17β-estradiol), the receptor undergoes a conformational change that alters the distance between Exo II and Exo IV and hence indicates that the ligand induced a change in receptor conformation.

To screen for compounds that reduce GPR30 activity, GPR30-specific antibody/fluorochrome binding is carried out in the presence and absence of a candidate compound. Detection of an allosteric change in the presence of the compound compared to its shape (as detected by FRET or BRET) in the absence of the compound indicates that the compound alters the function of GPR30.

### GPR30 Antibodies

Disclosed herein are antibodies to GPR30 proteins, or fragments of GPR30 proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Preferably, the monoclonal antibody is IgG or IgM isotype. Furthermore, in humans, the light chain may be a kappa chain or a lambda chain. Reference herein to antibodies includes a reference to all such classes, subclasses and types of human antibody species.

An isolated GPR30-related protein serves as an antigen, or a portion or fragment thereof, and is used as an immunogen to generate antibodies that immunospecifically bind the antigen using standard techniques for polyclonal and monoclonal antibody preparation. The full-length protein, or antigenic peptide fragments of the antigen, are used as immunogens. An antigenic peptide fragment comprises at least 6 amino acid residues of the amino acid sequence of the full length protein, such as an amino acid sequence shown in SEQ ID NO: 2, SEQ ID NO: 3, SEQ ID NO: 4, or SEQ ID NO: 5, and encompasses an epitope thereof such that an antibody raised against the peptide forms a specific immune complex with the full length protein or with any fragment that contains the epitope. Preferably, the antigenic peptide contains at least 8, 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of a GPR30-related protein that is located on the surface of the protein, e.g., a hydrophilic region. A hydrophobicity analysis of the human GPR30-related protein sequence will indicate which regions of a GPR30-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, Proc. Nat. Acad. Sci. USA 78: 3824-3828, 1991; Kyte and Doolittle J. Mol. Biol. 157: 105-142, 1982, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

### Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256: 495, 1975. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro. The immunizing agent will typically include the protein antigen, a fragment thereof, or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001, 1984; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220, 1980. Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

### Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525, 1986; Riechmann et al., Nature, 332: 323-327, 1988; Verhoeyen et al., Science, 239:1534-1536, 1988), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596, 1992).

### Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227: 381, 1991; Marks et al., J. Mol. Biol., 222:581, 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. Bio/Technology 10, 779-783, 1992; Lonberg et al. Nature 368 856-859, 1994; Morrison, Nature 368, 812-13, 1994; Fishwild et al, Nature Biotechnology 14, 845-51, 1996; Neuberger Nature Biotechnology 14, 826 1996; and Lonberg and Huszar Intern. Rev. Immunol. 13: 65-93, 1995.

Human antibodies may additionally be produced using transgenic nonhuman animals which are modified so as to produce fully human antibodies rather than the animal's endogenous antibodies in response to challenge by an antigen. (See PCT publication WO 94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305: 537-539, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210, 1986.

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al. (Science 229:81, 1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers (Kostelny et al., J. Immunol. 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al. (Proc. Natl. Acad. Sci. USA 90:6444-6448, 1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

The antibody of the invention is modified with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

### Immunoconjugates

Immunoconjugates containing an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate) are also within the scope of the invention.

Cytotoxic agents or enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

### Cancer/Tumor Stage

Cancer stage is determined, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Cancers or tumors are described according to tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Cancers and tumors of the invention are further described using histologic grade, also called differentiation, which refers to how much the tumor cells resemble normal cells of the same tissue type (see, National Cancer Institute, www.cancer.gov). Finally, cancers and tumors of the invention are described using nuclear grade, which refers to the size and shape of the nucleus in tumor cells and the percentage of tumor cells that are dividing (see, National Cancer Institute, www.cancer.gov).

The invention encompasses metastatic cancer, including the primary tumor and secondary sites of migration. Metastatic cancers and tumors of the invention are described as more severe, and consequently, as providing a less favorable prognosis if the tumor has secreted growth factors, degraded the extracellular matrix, become vascularized, lost adhesion to juxtaposed tissues, or further metastasized. Moreover, severity describes the number of locations to which a primary tumor has metastasized. Alternatively or in addition, severity includes the difficulty of treating tumors of varying types with respect to their locations. For example, inoperable tumors, those cancers which have greater access to multiple body systems (hematological and immunological tumors), and those which are the most resistant to traditional treatments are considered most severe.

As used herein, the term “treating” cancer is meant to describe a process of ameliorating or eliminating at least one sign or symptom of cancer. For example, cancer or tumor stage, grade, histological grade, and/or nuclear grade are signs of cancer. Conditions resulting either directly or indirectly from the activity of a tumor or cancer cell are considered symptoms of cancer for the purposes of the invention.

### EXAMPLES

### Example 1

### General Methods

Immunohistochemical analysis. GPR30 antibodies were generated in New Zealand white rabbits against a C-TER peptide (CAVIPDSTEQSDVRFSSAV; SEQ ID NO:7) comprising the carboxyl terminal 18 amino acid residues from the deduced amino acid sequence of human GPR30. Sera from immunized rabbits were affinity-purified on peptide columns before use. For GPR30 staining, formalin-fixed tissues were deparaffinized by heating slides to 60° C. for one hour followed by three consecutive extractions in Citrisolv (Fisher Scientific, Pittsburgh, Pa.). Tissues were then washed in ethanol, rehydrated and heated at 95° C. for 20 minutes in 0.1M sodium citrate, pH 6.0. Endogenous peroxidase activity was quenched in 3% H₂0₂ and nonspecific binding was blocked using bovine serum albumin. Slides were exposed to GPR30 peptide antibodies for 2 hours at ambient temperature and then washed three times in tris-buffered saline containing 0.05% Tween 20. Tissue-associated rabbit antibodies were detected using a dextran-coated polymer containing horseradish-peroxidase-conjugated goat anti-rabbit IgG (Envision-plus™) and diaminobenezidine as a substrate (Dako Cytomation, Carpinteria, Ca.). Nuclei were counterstained using Mayer's modified hematoxylin (PolyScientific, Bay Shore, N.Y.).

ER staining scores were determined by NCI-selected pathologists and were provided in the blind key that accompanied the CBCTR microarrays after submission of GPR30 results. For the purpose of showing representative examples of ER staining in the tumor microarray sets evaluated, ER was immunostained on a Dako Autostainer using the Envision-plus™ detection system.

Evaluation of the immunostaining pattern for GPR30. Two observers using a semiquantitative scoring system microscopically evaluated intensity, extent and subcellular distribution of GPR30. Scores were applied as follows: score 0: negative staining in all cells, score 1+: weakly positive or focally positive staining in less than 10% of the cells, score 2+: moderately positive staining covering 10% to 50% of the cells, and score 3+: strongly positive staining, including more than 50% of the cells. For statistical analysis as well as to reduce intraobserver variability, the immunohistochemical scores were further grouped in two categories: negative or weakly positive (0 and 1+) and moderately to strongly positive (+2 and +3). Patient data were derived from a blind key provided by the NCI after reporting GPR30 scores.

Statistical analysis. Associations between steroid receptor expression categories and tumor stage were evaluated using the Chi square test or the Fisher's exact test, as needed. Two parametric groups were compared using the student T test for independent samples. Comparison between two non-parametric (ordinal) groups was done using the Mann-Whitney U test. Two-tailed p values of 0.05 or less were considered to be statistically significant.

### Example 2

### GPR30/GPER-1 Predicts Reduced Overall Survival in Patients with Serous Ovarian Adenocarcinoma

The progress of ovarian carcinoma, the most lethal of all gynecological cancers, is grim, often with less than a two year life expectancy following surgical reduction of the tumor mass. Adjuvant therapy consists of platinum-based drugs and taxanes as ovarian carcinoma is often metastatic at first deduction and hormonal therapies targeted against the estrogen receptor, ER, yield favorable responses only in a small subset of patients. However, recent studies have rekindled an interest in the use of hormonal therapy to treat ovarian carcinoma due to evidence that aromatase inhibitors that block estrogen biosynthesis, have had benefit in patients that fail in chemotherapy.

The seven transmembrane receptor, GPR30/GPER-1 is linked to specific estrogen binding (Thomas et al. 2005. Endocrin 146:624-632; Revankar et al. 2005. Science 307:1625-30), estrogen release of membrane-tethered heparin bound-epidermal growth factor (HP-EGF) from breast cancer cells (Filardo et al. 2000. Molec Endrocrin 14:1649-1660) and the development of extramammary metastases (Filardo et al, 2006. Clin Cancer Res 12:6359-6366). To determine whether GPR30 also had significance in ovarian cancer, its expression in primary tumors and association with disease progression was evaluated.

Paraffin-embedded, formalin-fixed ovarian adenocarcinoma biopsy specimens representing different histopathelogical subtypes (serous, mucinous, endometrial and clear cell) from 78 patients that underwent surgery at Women and Infants Hospital between 1995-2000 were selected for study with IRB approval. All tumors studied were harvested at first diagnosis. GPR30 and ER expression was determined by immunohistochemical analysis and scored using a standard semiquantitative combinatorial scoring system based on staining intensity (scale of 1-3) and extent (scale of 1-3) and equally weighted for both criteria. The distribution pattern of estrogen receptors (ER and GPR30) was correlated with clinicopathelogical variables obtained during patient follow up.

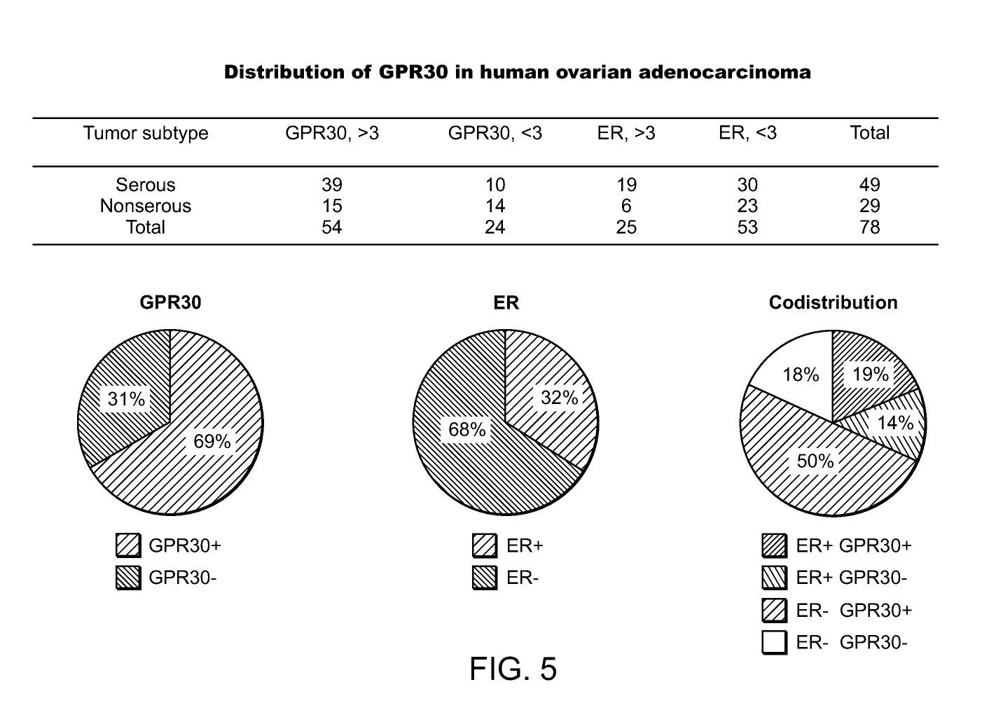

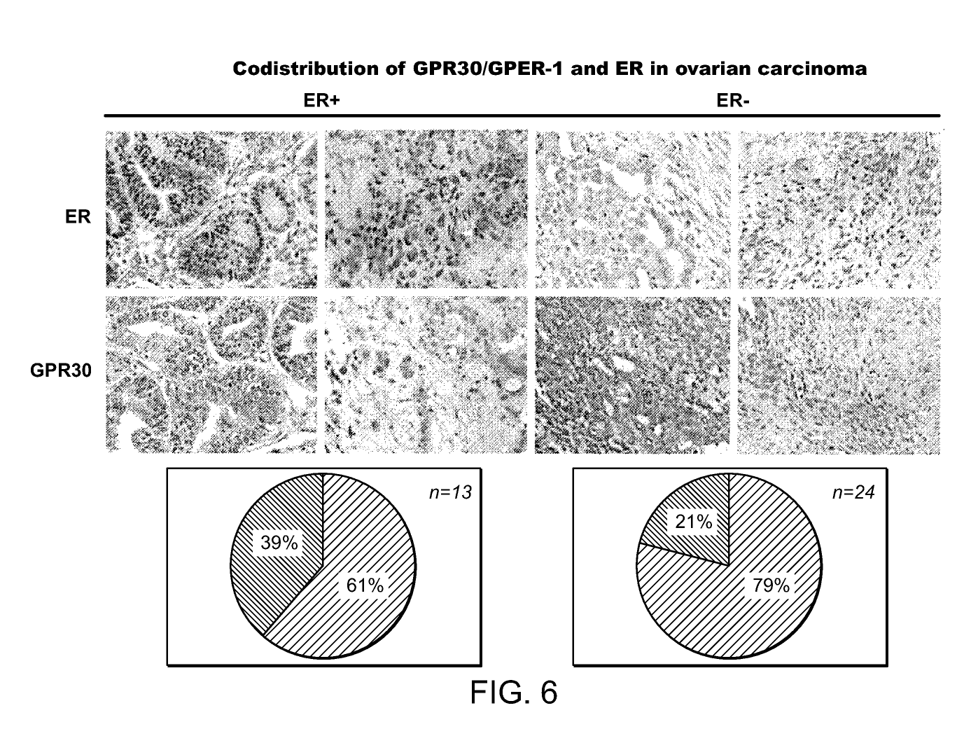

Distribution of GPR30 and ER in ovarian tumor tissue. Unlike normal ovarian epithelia, tumor tissue demonstrated variation in GPR30 staining with immunopositivity ranging from 0 to +9 (FIGS. 5 and 6). Biopsies exhibiting low GPR30 scores (<3) were considered negative, while tumor specimens that scored moderately or strongly (≧3) for GPR30 were categorized as positive. As observed in normal tissue, all GPR30 positive tumor biopsies exhibited a cytoplasmic staining pattern. In contrast, ER staining was confined to the nucleus (FIG. 6). Among 78 cases of ovarian carcinoma analyzed, approximately one-third (32%) demonstrated GPR30-positivity, with a significantly higher percentage of tumors expressing the alternative estrogen receptor, GPR30 (FIG. 5).

ER and GPR30 assort independently in ovarian carcinoma. While ER and GPR30 expression were independent variables, coexpression of GPR30 and ER was observed in approximately one in five tumors (19%, FIG. 5, right piechart). A roughly similar of tumors (14/78) expressed neither ER or GPR30 (18%, FIG. 5, right piechart). Interestingly, nearly 50% of the ER-positive tumors expressed GPR30 (FIG. 5, right piechart, 10/25) but a clear majority of ER-negative tumors (two-thirds) retained GPR30 (14%, FIG. 5, center piechart). These data indicate that a significant number of ovarian tumors that lack nuclear estrogen receptors main remain estrogen responsive by employing GPR30. Representative examples of human ovarian tumors with different estrogen receptor profiles are shown in FIG. 5.

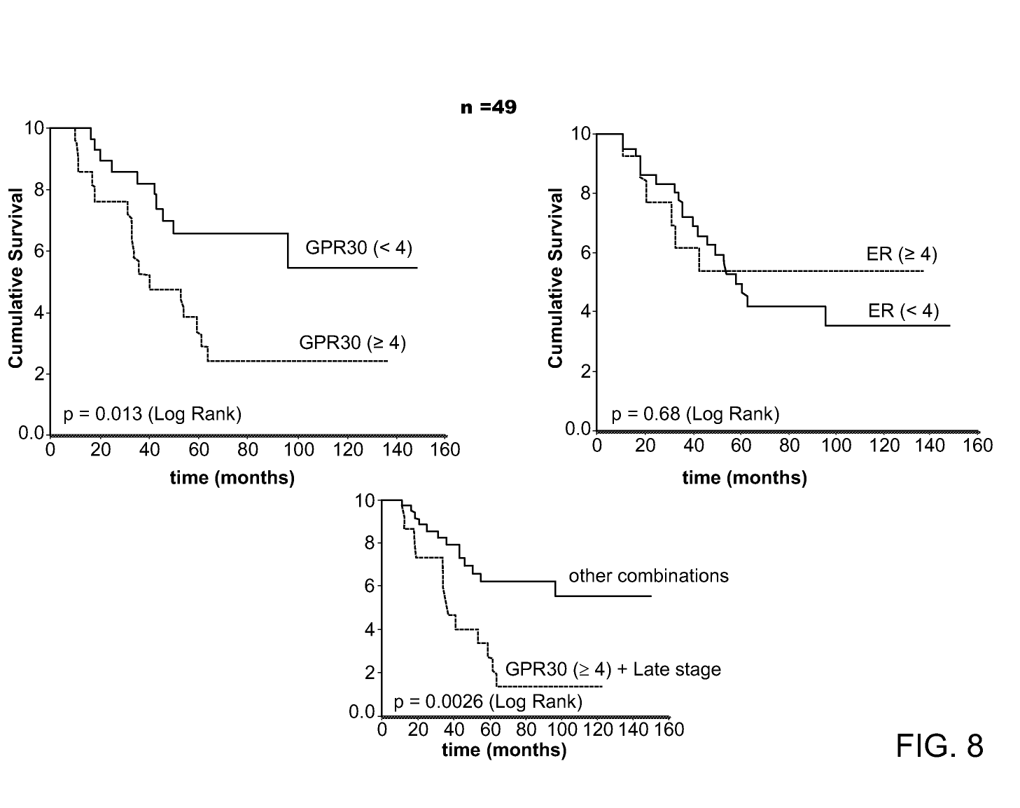

Association of GPR30 with overall survival of 5 years of follow-up. The relationship between GPR30 expression in primary serous ovarian carcinoma and overall patient survival during 60 months of follow-up was evaluated (FIG. 8). Using a Kaplan-Meier product limit method for analyzing patient survival, a significant difference (p=0.013) was observed in patients that were categorized based upon GPR30 expression levels in their primary tumors. Of the 28 patients whose tumors expressed low levels of GPR30 (<4), 54% were alive at the end of 60 months follow-up with 10 deaths and 18 censures during this time period (median survival time within this group was 40 months with 16 deaths and 5 censures. ER expression showed no significant difference in patient survival (p=0.68).

Tumor stage does not influence the predictive power of GPR30 on patient survival and each variable has an independent effect as determined by using a Cox Proportional hazard model (GPR30; p=0.02, Hazard, 2.6 vs. stage; p=0.05, Hazard 2.5).

Our data support the hypothesis that GPR30 has a significant biological role in human ovarian carcinoma, and they indicate that GPR30 and ER have independent influences on estrogen responsiveness in ovarian carcinoma.

### Example 3

### GPR30 in Primary and Metastatic Ovarian Carcinoma Correlates with Patient Survival

Biopsy specimens containing primary and/or metastatic ovarian cancer derived from patients at first diagnosis (prior to treatment) are immunostained with GPR30 peptide antibodies. GPR30 expression is evaluated blindly using a semiquantitative scoring system measuring staining intensity and extent, as described in Filardo et al. (2006. Clin Canc Res 12:6359-6366). Scored samples are then linked to clinicopathological data.

Affinity-purified antibodies specific for human GPR30 protein have been generated using a synthetic peptide CAVIPDSTEQSDVRFSSAV (SEQ ID NO: 8) derived from the carboxyl terminus of the human GPR30 polypeptide as immunogen and their specificity has been demonstrated as previously (Filardo et al. 2006. Clin Canc Res 12:6359-6366).

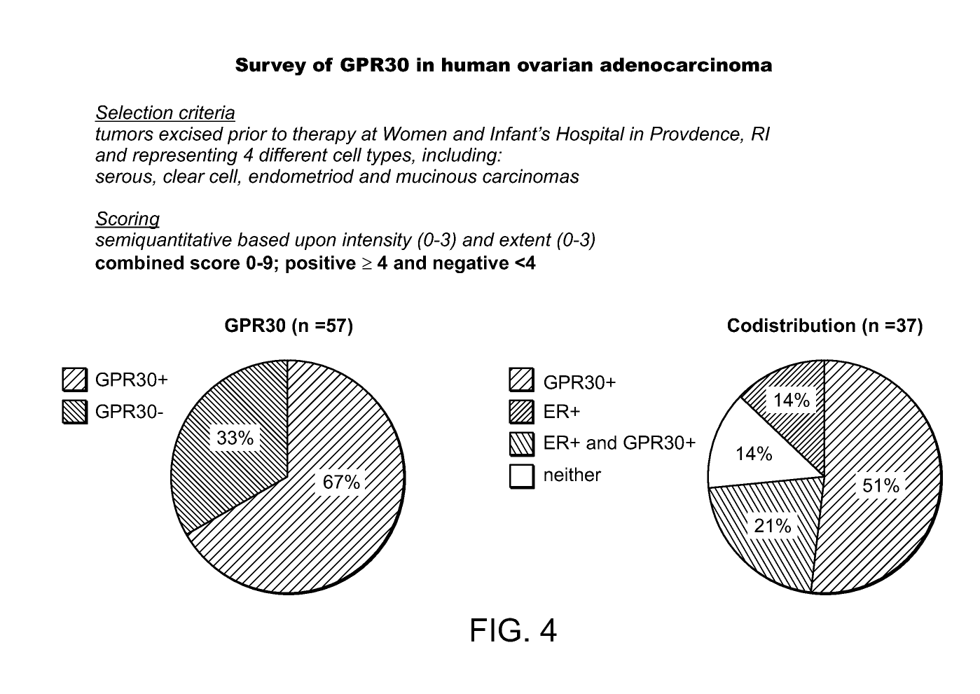

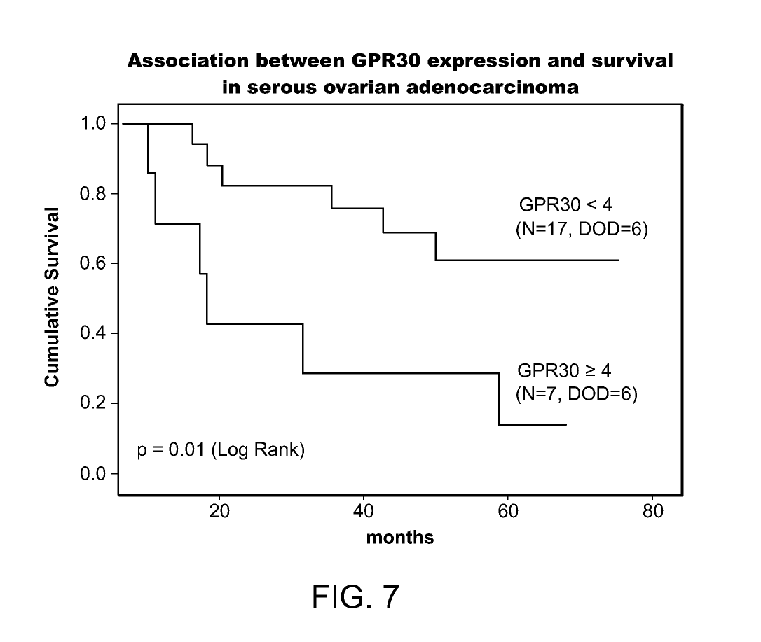

Preliminary analysis of 57 carcinomas indicates that GPR30 is expressed in two-thirds of cases (FIG. 4), and in 80% of serous tumors that lack ER (19 of 24) (FIG. 6). Of potential significance, a strong association was measured between tumors that express elevated GPR30 and overall patient survival at 5 years of follow-up (log rank=0.01) (FIG. 7).

Margaret Steinhoff, M D provides tissue blocks containing ovarian cancer and prepared from archival biopsy specimens cross-referenced from the WIH (Women and Infants Hospital) and Pathology Database and this study has been reviewed by the WIH Internal Review Board.

Immunostaining for ER and HER2/neu is performed as previously described (Filardo et al. 2006. Clin Canc Res 12:6359-6366). GPR30 staining is accomplished using horseradish peroxidase conjugated anti-rabbit IgG raised in goat with DAB as a substrate. Tissue is counterstained with Gillis' hematoxylin. Specimens are observed using a Nikon Microphot-FXA microscope and images are captured using a SenSys digital camera fitted with PVCAM acquisition software (Photometrics, Tucson, Ariz.) and analyzed using IPLab Spectrum 3.1. A single large batch of MCF-7 and MDAMB-231 breast cancer cells are included in staining and analysis to provide reference values for high and low GPR30 signals as well as for ER signals, respectively. Additional controls include an analysis of staining in adjacent, normal ovarian tissue. Medullary cells are negative while oocytes are positive. Dr Edmond Sabo (Department of Pathology, Technion University, Haifa, Israel) and Dr Margaret Steinhoff (Department of Pathology, WIH) score the tumor specimens along with at least one other observer. After scores have been compiled, the coded samples are matched to the patient data.

Correlation between expression of GPR30 and other molecular markers is evaluated using Spearman's coefficient of correlation. Association between categorical groups is done using the Chi-square test or Fisher's exact test as needed. Univariate analysis of survival is done using Kaplan-Meier curves and the log rank test. Multivariate survival analysis is done using the Cox proportional hazard model. Two tailed p-values of 0.05 or less, is considered statistically significant.

### Example 4

### Development of Human Ovarian Tumor Cell Models for Addressing the Influence of GPR30 on Tumor Growth, Progression and Responsiveness to Hormonal Therapy in Athymic Mice

Specific estrogen binding and estrogen signaling in breast cancer cells occurs independently of ER but rather requires GPR30 (Filardo, E. J. et al. 2000. Molec Endo. 14:1649-1660; Filardo, E. J. et al. 2002. Mol. Endocrinol. 16:70-84; Revankar, C. M. et al. 2005. Science 307:1625-1630). Preliminary analysis indicates that GPR30 and ER also assort independently in human ovarian tumor cells. Rapid GPR30 expression and rapid estrogen action is surveyed in ovarian cancer cell lines available through the NCI with different ER profiles. Endogenous GPR30 expression and function is restored or inhibited (by use of a dominant interfering mutant) in both ER-negative and ER-positive human ovarian cancer cell lines and the capacity of these cell lines to form tumor xenografts in athymic mice is tested under the influence of estrogenic hormones.

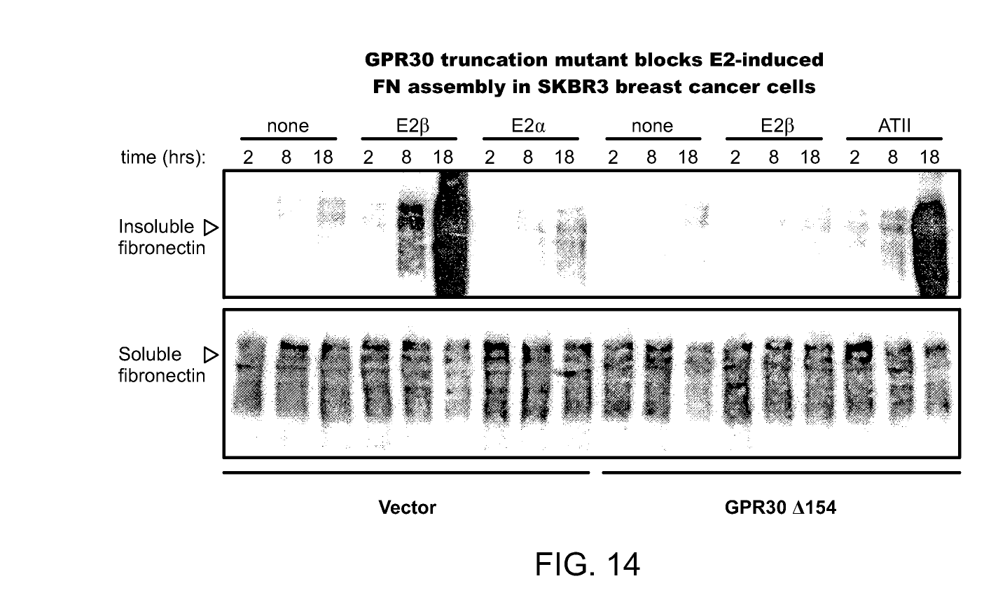

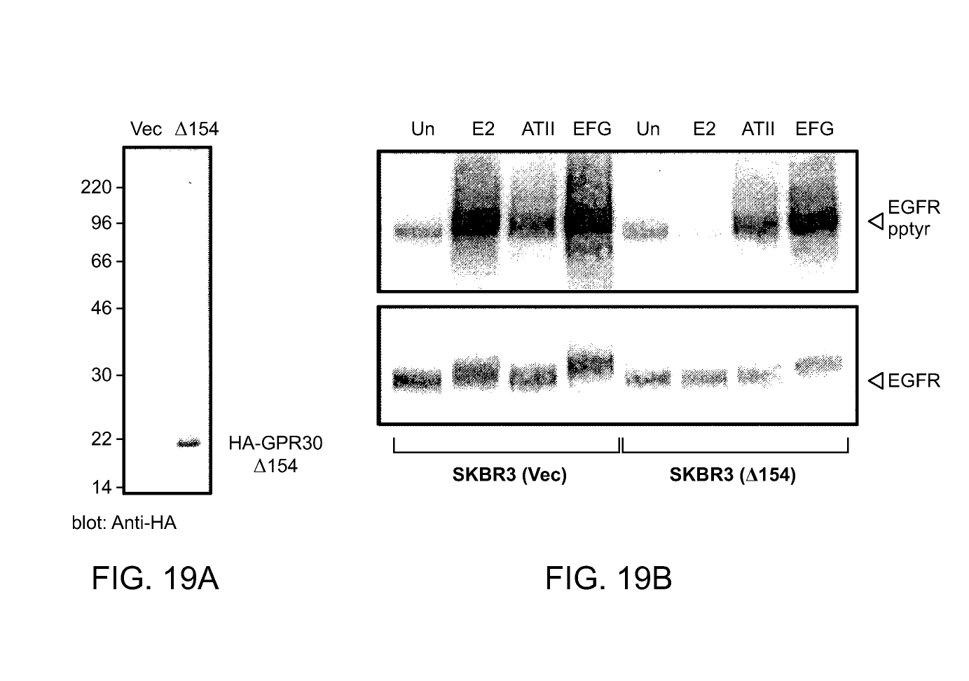

GPR30 peptide antibodies specifically detect GPR30 protein in Western blot analysis (Filardo, E. J. et al. 2006. Clin Cane Res 12:6359-6366). Culture conditions have been established to measure 17β-estradiolmediated activation of adenylyl cyclase (Filardo, E. J. et al. 2002. Mol. Endocrinol. 16:70-84) and EGFR activation (Filardo, E. J. et al. 2000. Molec Endo. 14:1649-1660) in breast cancer cells. A C-terminally truncated GPR30 has been generated that acts as an interfering mutant (FIGS. 19 and 14).

GPR30 expression and the capacity of estrogen to stimulate adenylyl cyclase or the EGFR is assessed by immunoblot analysis of detergent solubilized lysates prepared from serum-starved ovarian cancer cells that are left untreated or stimulated with estradiol, cholera toxin or EGF. GPR30 expression and activation of the EGFR is measured as described previously (Filardo, E. J. et al. 2000. Molec Endo. 14:1649-1660). Adenylyl cyclase activity is measured by employing phospho-specific antibodies for the cyclic AMP-regulated enhancer binding protein (CREB). Alternatively, adenylyl cyclase activity is measured in membrane homogenates by assaying cAMP in a competitve ELISA (Filardo, E. J. et al. 2002. Mol. Endocrinol. 16:70-84).

Forced overexpression of wild-type and mutant GPR30 in ovarian cancer cells. Ovarian cancer cells are transfected with cDNAs encoding wild-type or mutant GPR30 by employing cationic detergents as previously described (Filardo, E. J. et al. 2000. Molec Endo. 14:1649-1660).

### Influence of GPR30 on the Growth of Human Ovarian Tumor Xenografts.

Tumorigenicity studies. Experiments are conducted to determine the number of cells (105-107 cells) necessary to produce xenografts with 70-80% engraftment and a mean calculated weight of 50-100 mg within 7-14d. These experiments tumor cells are injected subcutaneously as well intraperitoneally into mice in the presence or absence of fibronectin. Exogenous E2 (or EGF) is administered via pellet implantation 2 weeks prior to tumor cell inoculation. The influence of ERα, ERβ and GPR30 (G1; Bologa et al, 2006) specific agonists and the ER antagonist, ICI 182, 780 (faslodex) on promoting tumor xenograft growth is also examined. At various points in time, after tumor cell implantation, mice are sacrificed and tumor growth is evaluated at the primary site or at distant sites including the surfaces of internal organs. Responsiveness of GPR30-positive and GPR30-negative ovarian tumor cell models to hormonal therapy (ER antagonist or the aromatase inhibitor, Arimidex) is tested as well.

Results. GPR30 action is expected to facilitate ovarian cancer cell growth or survival. It is further expected that interference with endogenous GPR30 activity in ovarian cancer cells attenuates tumor growth. In contrast, it is hypothesized that ectopic expression of GPR30 in ovarian cancer cells enhances the efficacy of tumor outgrowth and metastatic spread.

Adenylyl cyclase and EGFR function in ovarian cancer cells and positive internal controls are included in these experiments. While GPR30 is a Gs-coupled receptor in breast cancer cells, it may couple differentially to other G proteins in ovarian cells. Differential coupling of the same 7TMR to alternative G proteins has been described (Daaka et al, 1997) and this possibility is considered, particularly if GPR30 is present but does not signal to adenylyl cyclase or the EGFR.

Four outcomes are possible. (1) GPR30 expression occurs with rapid estrogen signaling. This outcome is further tested by the use of an interfering mutant to uncouple signaling. (2) Lack of GPR30 and absence of estrogen signaling. This result warrants determining whether receptor reconstitution results in signaling. Alternatively, rapid estrogen signaling (adenylyl cyclase and EGFR activity) may not be linked with GPR30 expression which gives rise to the final two possible outcomes. (3) GPR30 is present but does not signal. As discussed above, additional signaling endpoints (eg. calcium, phosphatidylinositol) are evaluated. Estrogen binding is also confirmed by radioreceptor assay (Thomas, P. et al. 2005. Endocrinol 146:624-632). (4) Rapid estrogen signaling occurs in the absence of GPR30. This finding suggests that receptors other than GPR30 promote estrogen signaling in ovarian cancer cells. It is worth mentioning that many ligands for 7TMRs, employ more than one related 7TMRs and is well illustrated by the fact that six related receptors comprise the adrenoceptor family. In this case, dominant interfering mutants may affect related receptors which also illustrates the value of using a dominant negative versus expression “knock-down” approaches.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of prognosis for ovarian adrenocarcinoma patients, comprising detecting GPR30 in a sample of ovarian tissue following excision of a primary tumor, wherein an elevation in the level of GPR30 compared to a normal control level or over time indicates presentation of late stage disease.

2. A method of prognosis for ovarian adrenocarcinoma patients, comprising detecting GPR30 and ER in a sample of ovarian tissue following excision of a primary tumor, wherein an elevation in the level of GPR30 and ER compared to a normal control level or over time indicates presentation of late stage disease.

3. The method of claim 1 or 2, wherein said late stage disease is stage II or III.

4. A method for predicting survival time of an ovarian adrenocarcinoma patient, comprising detecting GPR30 in a tissue biopsy, wherein an increase in GPR30 level is correlated with a decrease in survival time.

5. The method of claim 1, 2, or 3, wherein said primary tumor or tissue biopsy comprises serous, clear cell, endometrioid, or mucinous carcinoma cells.

6. A method for predicting the presence of distant metastatic neoplastic disease in a subject diagnosed as comprising a primary tumor, said method comprising detecting an increase in a GPR30 level in a tissue sample obtained from said primary tumor, wherein said increase indicates that said subject is suffering from or at risk of developing a malignant tumor at an anatomical site distant from said primary tumor.

7. The method of claim 6, further comprising detecting an increase in the integrin α5β1 level in a tissue sample obtained from said primary tumor, wherein said increase indicates that said subject is suffering from or at risk of developing a malignant tumor at an anatomical site distant from said primary tumor.

8. The method of claim 6 or 7, further comprising detecting an increase in the matrix adhesion molecule SNAKA51 level in a tissue sample obtained from said primary tumor, wherein said increase indicates that said subject is suffering from or at risk of developing a malignant tumor at an anatomical site distant from said primary tumor.

9. A method of prognosis for the presence of distant metastatic neoplastic disease in patients, comprising detecting GPR30 in a sample of ovarian tissue following excision of a primary tumor, wherein an elevation in the level of GPR30 compared to a normal control level or over time indicates enhanced fibrillogenesis.

10. A method of prognosis for the presence of distant metastatic neoplastic disease in patients, comprising detecting GPR30 in a sample of ovarian tissue following excision of a primary tumor, wherein an elevation in the level of GPR30 compared to a normal control level or over time indicates enhanced anchorage-independent growth.

11. A method of predicting the responsiveness of an ovarian tumor to hormonal therapy, comprising detecting GPR30 in a sample of ovarian tissue following excision of a primary tumor, wherein an elevation in the level of GPR30 compared to a normal control level or over time indicates an enhanced probability that hormonal therapy inhibits the tumor.

12. The method of claim 11, wherein said hormonal therapy comprises an aromatase inhibitor.

13. The method of claim 11 or 12, wherein said ovarian tumor comprises serous, clear cell, endometrioid, or mucinous carcinoma cells.

14. A method of predicting the responsiveness of a breast tumor to hormonal therapy, comprising detecting GPR30 in a sample of breast tissue, wherein an elevation in the level of GPR30 compared to a normal control level or over time indicates an enhanced probability that hormonal therapy inhibits the tumor.

15. The method of claim 14, wherein said breast tumor is characterized as ER-negative and GPR30-positive.
